(19) United States
(12) Patent Application Publication
(10) Pub. No.: US 2002/0173809 A1
(43) Pub. Date: Nov. 21, 2002

(54) Sutureless anastomosis system deployment concepts

(76) Inventors: Sidney D. Fleischman, Menlo Park, CA (US); Thomas H. Campbell, Brentwood, CA (US); Patrick M. Owens, Half Moon Bay, CA (US); Deborah Tolomeo, San Jose, CA (US); Rose Y. Leo, Hayward, CA (US)

Correspondence Address: Johney U. Han, Morrison & Foerster LLP, 755 Page Mill Road, Palo Alto, CA, 94304-1018, US

(21) Appl. No.: 10/096,263

(22) Filed: Mar. 8, 2002

Related U.S. Application Data
(63) Continuation of application No. 09/927,978, filed on Aug. 9, 2001.
(60) Provisional application No. 60/227,680, filed on Aug. 24, 2000.
(60) Provisional application No. 60/178,822, filed on Jan. 28, 2000.
(60) Provisional application No. 60/169,104, filed on Dec. 6, 1999.
(60) Provisional application No. 60/151,863, filed on Sep. 1, 1999.

(51) Int. Cl. A61B 17/08

(52) U.S. Cl. 606/153

(57) ABSTRACT

Sutureless anastomosis system deployment concepts are disclosed herein. Specifically, anastomosis strain relief devices are disclosed which are disposed, at least partially, over an anastomosis bypass graft just proximal to an attachment site between a host vessel wall and the fitting. The strain relief devices provide additional support to the graft while preventing kinking of the graft, especially when it emanates at acute angles from the anastomosis site. Furthermore, the strain relief provides additional support to the graft during manipulations involved in inserting and attaching ends of the graft. The devices can have a variety of configurations, e.g., helical, zig-zag, etc., depending upon the desired functionality. The strain relief may also be either incorporated into the graft or placed exterior to the graft and bonded. Moreover, integrated fittings or collars may be incorporated into the strain relief to expand its functions.

### CROSS-REFERENCE TO RELATED APPLICATIONS

This application is related to the following U.S. Patent Applications: co-pending U.S. Provisional Patent Application Serial No. 60/178,822, filed Jan. 28, 2000 and entitled “Improved Anastomosis Systems”, co-pending U.S. Provisional Patent Application Serial No. 60/169,104, filed Dec. 6, 1999 and entitled “Improved Anastomosis Systems”, co-pending U.S. Provisional Patent Application Serial No. 60/151,863, filed Sep. 1, 1999 and entitled “Additional Sutureless Anastomosis Embodiments”, and co-pending U.S. patent application Ser. No. 09/329,503, filed Jun. 10, 1999 and entitled “Sutureless Anastomosis Systems”, each of which is incorporated herein in its entirety by reference.

### FIELD OF THE INVENTION

This invention relates to devices for deploying and securing the ends of bypass grafts designed to provide a fluid flow passage between at least two host vessel regions (or other tubular structure regions). More particularly, the invention relates to bypass grafts that are secured at target host vessel locations thereby producing a fluid flow passage from the first host vessel location through the bypass graft and to the second host vessel location. The bypass grafts and deployment systems of the invention do not require stopping or re-routing blood flow to perform an anastomosis between a bypass graft and a host vessel. Accordingly, this invention describes sutureless anastomosis systems that do not require cardiopulmonary bypass support when treating coronary artery disease.

Current techniques for producing anastomoses during coronary artery bypass grafting procedures involve placing the patient on cardiopulmonary bypass support, arresting the heart, and interrupting blood flow to suture, clip, or staple a bypass graft to the coronary artery and aorta; cardiopulmonary bypass support is associated with substantial morbidity and mortality. The embodiments of the invention position and secure bypass grafts at host vessel locations without having to stop or re-route blood flow. Accordingly, the embodiments of the invention do not require cardiopulmonary bypass support and arresting the heart while producing anastomoses to the coronary arteries. In addition, the embodiments of the invention mitigate risks associated with suturing, clipping, or stapling the bypass graft to the host vessel(s), namely bleeding at the attachment sites and collapsing of the vessel around the incision point.

The invention addresses vascular bypass graft treatment regimens requiring end-side anastomoses to attach bypass grafts to host vessels. The scope of the invention includes improvements to the systems used to position and secure bypass grafts for treating vascular diseases such as atherosclerosis, arteriosclerosis, fistulas, aneurysms, occlusions, and thromboses. The improvements to the bypass grafts and delivery systems of the invention also aid in attaching the ends of ligated vessels, replacing vessels harvested for bypass grafting procedures (e.g. radial artery), and re-establishing blood flow to branching vessels which would otherwise be occluded during surgical grafting procedures (e.g. the renal arteries during abdominal aortic aneurysm treatment). In addition, the invention addresses other applications such as, but not limited to, producing arterial to venous shunts for hemodialysis patients, bypassing lesions and scar tissue located in the fallopian tubes causing infertility, attaching the ureter to the kidneys during transplants, and treating gastrointestinal defects (e.g. occlusions, ulcers, obstructions, etc.).

### BACKGROUND OF THE INVENTION

Stenosed blood vessels cause ischemia potentially leading to tissue infarction. Conventional techniques to treat partially or completely occluded vessels include balloon angioplasty, stent deployment, atherectomy, and bypass grafting.

Coronary artery bypass grafting (CABG) procedures to treat coronary artery disease have traditionally been performed through a thoracotomy with the patient placed on cardiopulmonary bypass support and using cardioplegia to induce cardiac arrest. Cardiac protection is required when performing bypass grafting procedures associated with prolonged ischemia times. Current bypass grafting procedures involve interrupting blood flow to suture or staple the bypass graft to the host vessel wall and create the anastomoses. When suturing, clipping, or stapling the bypass graft to the host vessel wall, a large incision is made through the host vessel and the bypass graft is sewn to the host vessel wall such that the endothelial layers of the bypass graft and vessel face each other. Bypass graft intima to host vessel intima apposition reduces the incidence of thrombosis associated with biological reactions that result from blood contacting the epithelial layer of a harvested bypass graft. This is especially relevant when using harvested vessels that have a small inner diameter (e.g. ≦2 mm).

Less invasive attempts for positioning bypass grafts at target vessel locations have used small ports to access the anatomy. These approaches use endoscopic visualization and modified surgical instruments (e.g. clamps, scissors, scalpels, etc.) to position and suture the ends of the bypass graft at the host vessel locations. Attempts to eliminate the need for cardiopulmonary bypass support while performing CABG procedures have benefited from devices that stabilize the motion of the heart, retractors that temporarily occlude blood flow through the host vessel, and shunts that re-route the blood flow around the anastomosis site. Stabilizers and retractors still require significant time and complexity to expose the host vessel and suture the bypass graft to the host vessel wall. Shunts not only add to the complexity and length of the procedure, but they require a secondary procedure to close the insertion sites proximal and distal to the anastomosis site.

Attempts to automate formation of sutureless anastomoses have culminated into mechanical stapling devices. Mechanical stapling devices have been proposed for creating end-end anastomoses between the open ends of transected vessels. Berggren et al. propose an automatic stapling device for use in microsurgery (U.S. Pat. Nos. 4,607,637, 4,624,257, 4,917,090, and 4,917,091). This stapling device has mating sections containing pins that are locked together after the vessel ends are fed through lumens in the sections and everted over the pins. This stapling device maintains intima-to-intima apposition for the severed vessel ends but has a large profile and requires impaling the everted vessel wall with the pins. Sakura describes a mechanical end-end stapling device designed to reattach severed vessels (U.S. Pat. No. 4,214,587). This device has a wire wound into a zigzag pattern to permit radial motion and contains pins bonded to the wire that are used to penetrate tissue. One vessel end is everted over and secured to the pins of the end-end stapling device, and the other vessel end is advanced over the end-end stapling device and attached with the pins. Sauer et al. proposes another mechanical end-end device that inserts mating pieces into each open end of a severed vessel (U.S. Pat. No. 5,503,635). Once positioned, the mating pieces snap together to bond the vessel ends. These end-end devices are amenable to reattaching severed vessels but are not suitable to producing end-end anastomoses between a bypass graft and an intact vessel, especially when exposure to the vessel is limited.

Mechanical stapling devices have also been proposed for end-side anastomoses. These devices are designed to insert bypass grafts, attached to the mechanical devices, into the host vessel through a large incision and secure the bypass graft to the host vessel. Kaster describes vascular stapling apparatus for producing end-side anastomoses (U.S. Pat. Nos. 4,366,819, 4,368,736, and 5,234,447). Kaster's end-side apparatus is inserted through a large incision in the host vessel wall. The apparatus has an inner flange that is placed against the interior of the vessel wall, and a locking ring that is affixed to the fitting and contains spikes that penetrate into the vessel thereby securing the apparatus to the vessel wall. The bypass graft is itself secured to the apparatus in the everted or non-everted position through the use of spikes incorporated in the apparatus design.

U.S. Surgical has developed automatic clip appliers that replace suture stitches with clips (U.S. Pat. Nos. 5,868,761, 5,868,759, and 5,779,718). These clipping devices have been demonstrated to reduce the time required when producing the anastomosis but still involve making a large incision through the host vessel wall. As a result, blood flow through the host vessel must be interrupted while creating the anastomoses.

Gifford et al. provides end-side stapling devices (U.S. Pat. No. 5,695,504) that secure harvested vessels to host vessel walls maintaining intima-to-intima apposition. This stapling device is also inserted through a large incision in the host vessel wall and uses staples incorporated in the device to penetrate into tissue and secure the bypass graft to the host vessel.

Walsh et al. propose a similar end-side stapling device (U.S. Pat. Nos. 4,657,019, 4,787,386, and 4,917,087). This end-side device has a ring with tissue piercing pins. The bypass graft is everted over the ring; then, the pins penetrate the bypass graft thereby securing the bypass graft to the ring. The ring is inserted through a large incision created in the host vessel wall and the tissue piercing pins are used to puncture the host vessel wall. A clip is then used to prevent dislodgment of the ring relative to the host vessel.

The end-side stapling devices previously described require insertion through a large incision, which dictates that blood flow through the host vessel must be interrupted during the process. Even though these and other clipping and stapling end-side anastomotic devices have been designed to decrease the time required to create the anastomosis, interruption of blood flow through the host vessel increases the morbidity and mortality of bypass grafting procedures, especially during beating heart CABG procedures. A recent experimental study of the U.S. Surgical One-Shot anastomotic clip applier observed abrupt ventricular fibrillation during four of fourteen internal thoracic artery to left anterior descending artery anastomoses in part due to coronary occlusion times exceeding 90 seconds (Heijmen et al. “A Novel One-Shot Anastomotic Stapler Prototype for Coronary Bypass Grafting on the Beating Heart: Feasibility in the Pig.” J Thorac Cardiovasc Surg. 117:117-25; 1999).

A need thus exists for bypass grafts and delivery systems that are capable of quickly producing an anastomosis between a bypass graft and a host vessel wall without having to stop or reroute blood flow. These anastomoses must withstand the pressure exerted by the pumping heart and ensure blood does not leak from the anastomoses into the thoracic cavity, abdominal cavity, or other region exterior to the vessel wall.

### SUMMARY OF THE INVENTION

The embodiments of the present invention provide improvements to the anastomosis systems that enable a physician to quickly and accurately secure a bypass graft to a host vessel or other tubular body structure. The deployment processes of the invention do not require stopping or re-routing blood flow while producing the anastomosis; conventional techniques require interrupting blood flow to suture, clip, or staple a bypass graft to the host vessel wall.

The fittings of the invention are intended to secure biological bypass grafts, obtained by harvesting vessels from the patient or another donor patient, or synthetic bypass graft materials to a patients host vessel. When using harvested vessels, the fitting embodiments must accommodate a variety of harvested vessel sizes and wall thicknesses. When using synthetic bypass graft materials, the fittings may be incorporated in the bypass graft design to eliminate the step of attaching the bypass graft to the fitting prior to deploying the bypass graft and fitting.

One aspect of the invention involves enhanced deployment components that facilitate inserting the bypass graft and fitting combination through an opening in the host vessel wall. In particular, the invention details punching dilators capable of coring a section of tissue and positioning an access sheath through the aperture defined by the punch. These punching dilators are capable of accessing the interior surface of the host vessel wall, punching an aperture through the host vessel wall, and positioning an access sheath through the opening without having to stop or re-route blood flow through the host vessel.

Additional sheathless anastomosis embodiments are also disclosed which are designed to insert the petals or securing end of the end-side fitting into the host vessel without having to insert the fitting through an access sheath. As such the maximum expanded diameter of the opening through the host vessel wall is limited to that required to insert the petals or other securing end of the end-side fitting. Therefore, hemostasis is improved at the interface between the fitting and the opening through the host vessel wall.

Also included in the invention are compressible, expandable end-side fittings that have the strain relief integrally attached to the base of the fitting. These embodiments facilitate securing the bypass graft to the stem of the fitting and ensure the bypass graft remains bonded to the base of the fitting while the base (and bypass graft) is compressed into a reduced diameter for insertion through a smaller diameter access sheath or other deployment device.

The invention also describes enhancements to the overall system to continue to make the anastomosis approach amenable to less invasive procedures, such an endoscopic, port access approaches. To accomplish this, additional components, primarily focused on exposing the host vessel wall, are included in the complete system. In particular, incising devices configured to cut individual layers of tissue to expose the underlying tissue and prevent unwanted damage to the underlying tissue are disclosed. These incising devices are configured to access the pericardial space by cutting the parietal pericardium without cutting into the heart. Alternatively, these incising devices may be used to create longitudinal incisions through the host vessel wall or separate the host vessel from adjacent anatomy (e.g. dissecting the coronary artery from the heart).

Further features and advantages of the inventions will be elaborated in the detailed description and accompanying drawings.

### BRIEF DESCRIPTION OF THE DRAWINGS

FIGS. 1A and 1B are side-sectional views of an over-the-wire punching dilator of the present invention.

FIGS. 1C and 1D are side-sectional views of an alternative over-the-wire punching dilator of the present invention.

FIGS. 2A and 2B are side-sectional views of another over-the-wire punching dilator of the present invention.

FIGS. 3A and 3B are side-sectional views of an angled over-the-wire punching dilator of the present invention.

FIGS. 4A to 4D are side-sectional views of a combination incising and punching dilator of the present invention.

FIGS. 4E and 4F are side views of two distal incising section embodiments of the combination incising and punching dilator of the present invention.

FIG. 4G is an end view of the distal incising section of FIG. 4F.

FIGS. 5A to 5D are side-sectional views of a compressible, expandable punching dilator of the present invention.

FIGS. 5E and 5F are side views of an alternative compressible, expandable punching dilator of the present invention.

FIGS. 6A to 6C are side-sectional views of yet another compressible, expandable punching dilator of the present invention.

FIG. 7A is a side-sectional view of an alternative incising and punching dilator of the present invention.

FIGS. 7B to 7D show incising and punching dilators that incorporate electrodes of the present invention to cauterize or coagulate the vessel tissue.

FIGS. 8A and 8B are side-sectional views of a screw-in coring device of the present invention.

FIGS. 8C and 8D are side-sectional views of an alternative screw-in coring device of the present invention.

FIG. 9A is a side-sectional view of a handle incorporating a movable actuation mechanism of the present invention.

FIG. 9B is a side-sectional view of an alternative handle of the present invention.

FIGS. 10A and 10B are a side view and an end view of a punching dilator of the present invention that incorporates a screw-in distal section.

FIGS. 10C to 10E are side views of a shearing punch dilator of the present invention.

FIGS. 10F and 10G are perspective and side views, respectively, of an alternative distal movable section for a shearing punch dilator of the present invention.

FIG. 10H shows an assembled punching dilator of the present invention.

FIGS. 11A and 11B show two potential punch geometries created with the punching dilator of the present invention.

FIGS. 12A and 12B are side and end views of a cutting element of the present invention used to create an oval punch.

FIG. 13A is a side view of a distal section used to create an oval punch.

FIGS. 13B and 13C are end views of the distal section of FIGS. 13A.

FIG. 14 shows a dilator of the present invention that incorporates a stop and a distal extension to enable remote manipulation.

FIG. 15 shows a pre-split access sheath of the present invention that incorporates a clamping mechanism to restrain the two halves of the access sheath in a closed position.

FIG. 16 is a flattened view of a pre-split loading sheath of the present invention.

FIG. 17 is a stretched and flattened view of a strain relief of the present invention.

FIG. 18 is a perspective view of a screw-in fitting of the present invention.

FIG. 19A is a perspective view of a dilating fitting of the present invention.

FIG. 19B is a flattened view of a compressible, expandable dilating fitting of the present invention.

FIG. 20 is a perspective view of a deployment device of the present invention used in conjunction with the dilating fitting of FIG. 19A.

FIG. 21 is a flattened view of an alternative dilating fitting of the present invention.

FIGS. 22A and 22B are perspective views of alternative strain relief embodiments of the present invention.

FIGS. 23A and 23B are a flattened views of a compressible, expandable fitting of the present invention that incorporates a strain relief.

FIG. 24 is a flattened view of another compressible, expandable fitting of the present invention that incorporates a strain relief.

FIGS. 25A and 25B are perspective views of two collars, or compression rings, of the present invention.

FIGS. 26A and 26B are top view and perspective views, respectively, of an alternative collar of the present invention.

FIGS. 26C and 26D are perspective views showing the deployment of the collar of FIGS. 26A and 26B.

FIG. 27 shows a strain relief of the present invention that also functions as a collar.

FIGS. 28A to 28C are side-sectional views of a vacuum assisted incisor of the present invention.

FIGS. 29A to 29C are side-sectional views of a vacuum assisted scalpel of the present invention.

FIG. 30 is a side-sectional view of an incising device of the present invention.

FIGS. 31A and 31B are end and side views, respectively, of a loading aid of the present invention.

### DETAILED DESCRIPTION OF THE INVENTION

The systems of the invention are intended to produce anastomoses between bypass grafts and host vessels to treat vascular abnormalities such as stenoses, thromboses, other occlusions, aneurysms, fistulas, or other indications requiring a bypass graft. The systems of the invention are also useful in bypassing stented vessels that have restenosed, and saphenous vein bypass grafts that have thrombosed or stenosed. Current approaches for treating stenosed stents have not been successful at safely and reliably removing the occlusion and opening the vessel lumen. Therefore the approach described by this invention, which produces a blood flow conduit around the stented lesion, mitigates concerns associated with damaging the stent or forming emboli while removing deposits attached to the stent. The approach may also be used to re-establish blood flow during redo procedures when the saphenous vein grafts (or other bypass grafts) have restenosed or thrombosed.

The embodiments of the invention also provide mechanisms to secure branching vessels to a replacement graft during surgical procedures in which the branching vessels would otherwise be occluded from blood flow (e.g. reattaching the renal arteries, mesenteric artery, celiac artery, and intercostal arteries during treatment of abdominal aortic aneurysms that are pararenal, suprarenal, or thoracoabdominal in classification). The embodiments of the invention also enable reattaching the left main artery and right coronary artery during aortic root replacement procedures. Similarly, the connectors of the invention may be utilized in reinforcing the coronary artery ostia when treating ostial stenoses.

The fitting and delivery system embodiments discussed in this invention are directly amenable to robotic surgery and less invasive (i.e. minimally invasive) surgery involving a thoracostomy or mini median sternotomy to access the anastomosis site, and endoscopes to visualize the thoracic cavity while producing the anastomoses. In particular, the fittings and delivery system embodiments of the invention enable automating the attachment of the bypass graft to the fitting, especially when considering the use of the loading sheath and/or end-side fittings capable of being advanced over a guidewire or along another guide member, as described below. In addition, the deployment and securing systems of the invention are significantly easier to automate than conventional suturing.

Bypass Grafts

The bypass graft may be a synthetic graft material, harvested vessel, other tubular body structure, or other flat body structure that may be rolled into a tube, depending on the indication for use. The harvested vessels may be an internal mammary artery, mesenteric artery, radial artery, saphenous vein or other body tubing. Harvested vessels may be dissected using newer minimally invasive, catheter-based techniques or standard surgical approaches. Flat body structures may be pericardium, or other anatomic structure that may be harvested, flattened, rolled, and affixed into a tube. The end-side fittings in accordance with the invention are designed to attach bypass grafts to host vessels (or other tubular structures). The fittings used to position and attach such bypass grafts are extensions of the collet and grommet embodiments described in U.S. Pat. No. 5,989,276 to Houser et al., and the fittings described in U.S. patent application Ser. No. 09/329,503, both of which are incorporated herein by reference. The primary advantage of biological bypass grafts (e.g. harvested vessels) over currently available synthetic materials is the reduction in thrombosis especially when using small diameter (e.g. ≦2 mm) bypass grafts. However, the fittings and delivery systems of the invention are equally effective at positioning and securing all types of bypass grafts, biological and synthetic.

Synthetic bypass grafts may be manufactured by extruding, injection molding, weaving, braiding, or dipping polymers such as polytetrafluoroethylene (PTFE), expanded PTFE, urethane, polyamide, polyimide, nylon, silicone, polyethylene, collagen, polyester, polyethylene terephthalate (PET), composites of these representative materials, or other suitable graft material. These materials may be fabricated into a sheet or tubing using one or a combination of the stated manufacturing processes. The sides of sheet materials may be bonded using radiofrequency energy, laser welding, ultrasonic welding, thermal bonding, sewing, adhesives, or a combination of these processes to form tubing. The synthetic bypass graft may be coated, deposited, or impregnated with materials, such as parylene, heparin solutions, hydrophilic solutions, thromboresistance substances (e.g., glycoprotein IIb/IIIa inhibitors), antiproliferative substances (e.g., Rapamycin), or other substances designed to reduce thrombosis or mitigate other risks that potentially decrease the patency of synthetic bypass grafts. In addition, synthetic bypass grafts may be seeded with endothelial cells, or other biocompatible materials that further make the inner surface of the bypass graft biologically inert.

The primary advantage of synthetic bypass graft materials is the ability to bond the bypass graft to the fittings prior to starting the procedure or incorporate the fittings into the bypass graft by injection molding, adhesive bonding, or other manufacturing process. Currently, synthetic bypass grafts are indicated for blood vessels having medium and large diameters (e.g. >3 mm), such as peripheral vessels, tubular structures such as the fallopian tubes, or shunts for hemodialysis. However, medical device manufacturers such as Thoratec Laboratories, Inc. are evaluating synthetic bypass grafts for coronary indications. In this disclosure and the accompanying drawings, reference to bypass graft may pertain to either biological bypass grafts such as harvested vessels or synthetic bypass grafts, unless specifically stated.

As discussed in co-pending U.S. patent application Ser. No. 08/932,566, filed Sep. 19, 1997 and U.S. Pat. No. 5,989,276, both of which are incorporated in their entirety herein by reference, support members may be associated with the graft. The support members may be laminated between layers of graft material, reside on the interior of the graft, reside against the exterior surface of the graft, or weave through the graft between the inside and outside surfaces. When using synthetic grafts, the support members are preferably laminated between layers of graft material. The synthetic graft encompassing support members may be fabricated by extruding, spraying, injection molding, or dipping a primary layer of graft material over a removable mandrel; positioning, winding or braiding the support members on the primary layer; and extruding, spraying, injection molding, or dipping a secondary layer over the graft material/support member combination. Alternatively, the support members may be encompassed between layers of graft material that are sintered together; this technique is especially pertinent when using expanded PTFE graft material. The support members may be fabricated from a metal, alloy (e.g. stainless steel or nickel titanium), or polymer (e.g. nylon or polyester); however, the support members preferably have a shape memory and exhibit superelastic properties. Support members enhance the performance of the bypass graft by maintaining lumenal patency, increasing the burst strength, preventing graft kinking, maintaining flexibility, and increasing the column strength. Support members fabricated from superelastic alloys, such as nickel titanium, provide additional reinforcing of the bypass graft and/or vessel wall and prevent permanent deforming upon exposure to external forces. Such support members also permit compressing the bypass graft into a low profile during deployment through the host vessel wall; the support members urge the bypass graft to expand towards its preformed configuration after the external force (e.g. delivery system) is removed.

End-Side Fittings

The fittings consist of one or more components designed to secure a bypass graft to the fitting and the fitting to the host vessel wall to produce a fluid tight bond between the bypass graft and the host vessel. The fittings may be used to produce end-side anastomoses for medium and small diameter vessels (e.g. upper and lower extremity vessels, and coronary vessels) where retrograde blood flow is essential, and end-side anastomoses for large diameter vessels (e.g. the aorta, and iliac artery). The fittings and delivery systems described below may be modified to accommodate end-end anastomoses by reducing, reshaping, or eliminating the petals from the design.

The end-side fittings are constructed from a metal (e.g. titanium), alloy (e.g. stainless steel or nickel titanium), thermoplastic (e.g. PTFE), thermoset plastic (e.g. polyethylene terephthalate, or polyester), silicone or combination of the aforementioned materials into a composite structure; other materials may alternatively be used. For example, end-side fittings fabricated from nickel titanium may be clad with expanded PTFE, polyester, PET, or other material that may have a woven or porous surface. The fittings may be coated with materials such as parylene or other hydrophilic substrates that are biologically inert and reduce the surface friction. To further reduce the surface tension, metallic or metallic alloy fittings may be electropolished. Evidence suggests that electropolishing reduces platelet adhesion because of the smooth surface. Alternatively, the fittings may be coated with heparin, thromboresistance substances (e.g. glycoprotein IIb/IIIa inhibitors), antiproliferative substances (e.g. Rapamycin), or other coatings designed to prevent thrombosis, hyperplasia, platelet aggregation, or restenosis around the attachment point between the bypass graft and the host vessel. Alternatively, a material such as platinum, gold, tantalum, tin, tin-indium, zirconium, zirconium alloy, zirconium oxide, zirconium nitrate, phosphatidyl-choline, pyrolytic carbon, or other material, may be deposited onto the fitting surface using electroplating, sputtering vacuum evaporation, ion assisted beam deposition, vapor deposition, silver doping, boronation techniques, a salt bath, or other coating process. A still further improvement of the fittings is to include beta or gamma radiation sources on the end-side fittings. A beta or gamma source isotope having an average half-life of approximately 15 days such as Phosphorous 32 or Palladium 103 may be placed on the base and/or petals of the end-side fitting using an ion-implantation process, chemical adhesion process, or other suitable method.

End-side fitting embodiments may be fabricated from a tube of material having a desired cross-sectional geometry. The desired pattern of petals, tabs, holes, slots, and spaces may be fabricated on the tubular metal material and may be formed using chemical etching, electrical discharge machining (EDM), laser cutting, or other manufacturing process. These end-side fittings may be maintained as a complete tube or may be fabricated to make the fitting compressible and expandable.

Alternatively, the end-side fitting embodiments may be fabricated from a sheet of material cut into the desired pattern and formed (e.g. through an annealing process) into the desired cross-sectional geometry (circular, elliptical, or other shape). The sides of the fitting may be bonded to form an enclosed tube or may be formed with a gap between opposite sides to enable compressing the fitting into a reduced diameter for positioning the bypass graft over the base of the fitting and inserting the fitting through an opening into a host vessel having a diameter less than the expanded diameter of the fitting. Such compressible fittings also facilitate sizing issues since they accommodate a wide range of bypass graft sizes.

To produce these end-side fittings, the raw material may be fabricated into the desired pattern by chemically etching, EDM, laser cutting, or other manufacturing process. End-side fittings fabricated from sheet stock are then wrapped around mandrels having the desired resting cross-sectional profile(s) and the end-side fitting is heated until it assumes this configuration. If the sides are to be bonded, spot welding, laser welding, or other manufacturing process may be employed.

When forming the resting configuration of the compressible and expandable split-wall end-side fitting, a gap is produced between opposite sides of the base or stem of the fitting. The gap between the sides of the fitting permits compressing the end-side fitting into a reduced diameter which facilitates positioning the bypass graft over the base of the fitting and/or advancing the fitting through a delivery system having an inner diameter less than the outer diameter of the fitting in its expanded, resting configuration. This split-wall end-side fitting is also expandable so it may be enlarged for advancing outside a bypass graft everted over or positioned over a central member. In this case, the split-wall end-side fitting secures the bypass graft against the central member. In addition, this enables using a single fitting configuration to accommodate a wide range of bypass graft sizes.

The base of the fitting (as well as the petals if desired) may be covered with a blood impervious, porous, compliant material such as silicone, urethane, expanded PTFE, PTFE, fluorinated ethylene propylene (FEP), polyester, PET, or other material. The covering over the base of the fitting may be fabricated with dipping, injection molding, sintering, cladding, or other manufacturing process. This covering enables compressing and expanding the base and/or petals of the fitting yet maintains the leak resistance of the anastomosis and isolates the cut end of the bypass graft from blood.

The petals in many of these fitting embodiments are shown generally straight (i.e. at an angle of about zero degrees from the base of the fitting). During manufacture, the petals may be thermally formed at any angle between about 30 and about 150 degrees from the base of the fitting such that the petals contact the interior surface of the host vessel once the fitting is inserted through the host vessel wall. The petals, having an angle between about 30 and about 150 degrees from the base of the fitting in their resting orientation, also compress into a reduced outer diameter during deployment through delivery system and expand towards their resting configuration once deployed inside the host vessel. Other angles are also possible. The number of petals incorporated in the end-side fitting design depends on the size of the bypass graft and the size of the host vessel. The number of petals also depends on the desired tensile strength between the fitting and the host vessel; increasing the number of petals in turn increases the force required to pull the fitting petals out of the host vessel. After advancing the fitting through an opening into the host vessel wall, the bypass graft and fitting combination is gently retracted to engage the interior vessel wall with the petals. For mechanical securing, a support device is advanced over and locked to the fitting thereby compressing the vessel wall against the petals.

After positioning the end-side fitting inside the vessel such that the base of the fitting extends through an opening into the host vessel wall and the petals contact the interior surface of the host vessel, the support device is positioned over the base of the fitting and locked in place. The end-side fittings may incorporate tabs, threads, or other locking mechanism with which to secure a support device to the end-side fitting. The support device is alternatively locked to the base of the fitting using adhesives, implantable clips, staples, sutures, or other attachment means.

The support device may be constructed from polyethylene, polyurethane, polycarbonate, thermoplastic (such as PEEK, manufactured by Victrex PLC, United Kingdom), silicone, nickel titanium, spring stainless steel, other alloys, combination of the aforementioned materials, or other materials that may be extruded, injection molded, rolled, or otherwise formed into a tube having the desired cross-sectional profile. In addition, the support device may incorporate a braided, woven, or wound layer laminated between two polymer layers to resist kinking and improve the column strength and torque response. Alternatively, the support device may be fabricated with a superelastic central layer encapsulated with a compliant covering. The support device preferably has porosity sufficient to permit air to diffuse into tissue covered by the support device. The pore size may be as high as approximately 100 μm as long as the porosity is chosen such that blood does not continually leak through the support device. If the pore size is chosen such that it completely restricts blood flow even when the porosity is extremely high then the pore size needs to be less than approximately 8 μm.

Deployment Systems

Conventional anastomosis techniques require a relatively large incision through the vessel wall and use sutures, commercially available clips, or stapling devices to bond the end of the bypass graft to the edges of the punch created in the vessel wall. In certain cases, the structural integrity of the vessel wall may be weakened causing the vessel to collapse at the anastomosis site, especially when the bypass graft is not appropriately aligned to the host vessel incision. Therefore, the deployment system embodiments of the invention are designed to quickly access the host vessel through a small puncture in the vessel wall. As such, the deployment systems are designed to prevent excess blood loss when accessing the host vessel and deploying the bypass graft and fitting combination, thereby eliminating the need to stop or re-route blood flowing through the host vessel. This approach also improves the leak resistance around the fitting due to elastic compression of the vessel wall around the fitting and automatically aligns the bypass graft to the host vessel wall at the anastomosis site.

For surgical applications, physicians are able to access the anastomosis sites from the exterior surface of the host vessel(s). The deployment system of the surgical approach must permit removal after both ends of the bypass graft are secured and the delivery system resides around the attached bypass graft. The deployment system leverages conventional intravenous (I.V.) access techniques to produce an opening through the host vessel wall. Guidewires have commonly been used to gain access into the host vessel after puncturing the host vessel wall with a needle. In addition, the technique of inserting a sheath into a host vessel by advancing it over a dilating mechanism and a guidewire is commonly used when performing the Seldinger technique during catheterization procedures.

The sheath and dilating mechanism of the deployment system, as previously described in U.S. Pat. No. 5,989,276, co-pending U.S. Provisional Patent Application Serial No. 60/151,863 and co-pending U.S. Provisional patent application Ser. No. 09/329,503 may be constructed from polyethylene, polycarbonate, PEEK, other polymer, metal, or metal alloy that may be extruded, injection molded, or swaged into a tube having the desired cross-sectional profile. A taper and radius may be formed in the components of the deployment system by thermally forming the tubing into the desired shape or incorporating such features in the injection molding cast. In addition, the components of the deployment system may incorporate a softer distal tip fabricated by thermally bonding a short section of lower durometer tubing to the sheath or tapering the thickness of the sheath tubing.

To prevent the backflow of blood through deployment sheaths, hemostatic valves may be used. The hemostatic valves prevent blood leakage but permit insertion of a device such as a fitting with an attached bypass graft through the sheath. The hemostatic valve of the delivery system of the invention also incorporates a mechanism to separate along at least one side and remove from around the bypass graft. To accomplish this, the hemostatic valve is attached to the hub of the sheath and includes a mechanism to separate along at least one side. To incorporate a splitting mechanism in the deployment sheath, at least one groove, series of perforations, slot, slit, or combination of these features are incorporated in the sheath tubing and hub member. The at least one groove, series of perforations, slot, slit, or combination of these features may be fabricated while injection molding or otherwise manufacturing the sheath tubing and/or hub, or may be formed in the assembled sheath by laser drilling, milling, or other manufacturing process.

Various configurations of deployment sheaths and associated deployment components are discussed in U.S. Pat. No. 5,989,276, co-pending U.S. Provisional Patent Application Serial No. 60/151,863, and co-pending U.S. patent application Ser. No. 09/329,503. Improvements to the operation of such deployment systems will be identified below and include mechanisms to relieve the stress around the opening through the host vessel, enable remote separation of splittable sheaths for removal from around the bypass graft, and preserve hemostasis during the deployment and securing processes.

Observations during experimental evaluations have demonstrated that over expansion of an opening through a host vessel wall potentially causes radial splitting of the host vessel wall, especially when over expanding small diameter vessels. To prevent this radial splitting, punching mechanisms are used to remove tissue thereby reducing the stress on the host vessel wall during expansion of the opening.

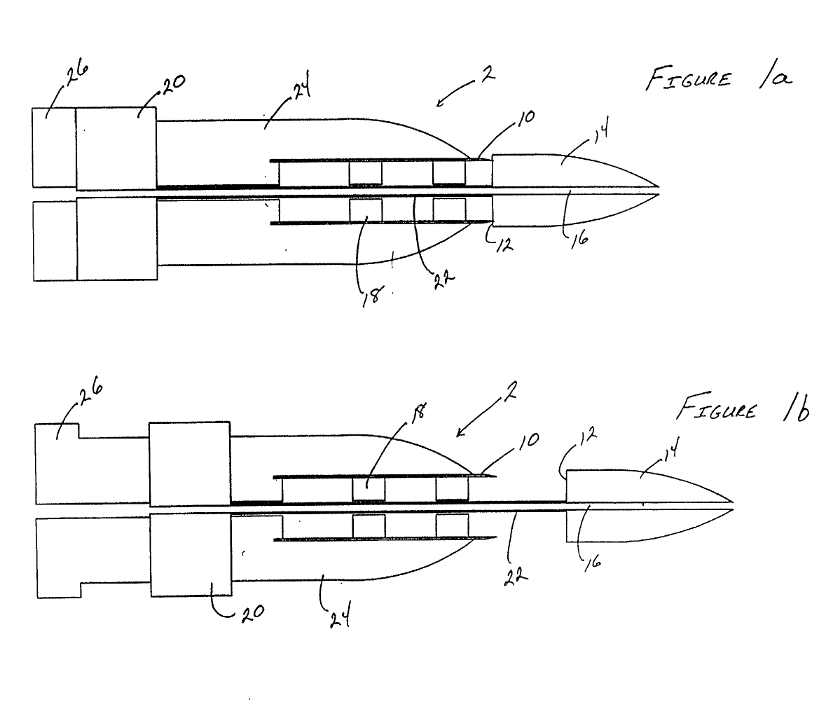

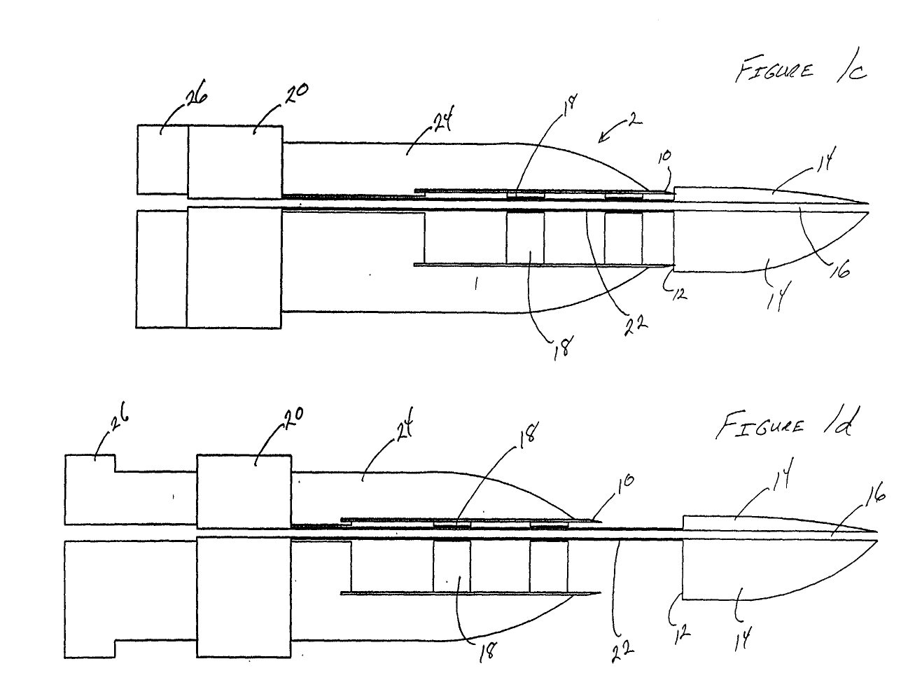

FIGS. 1A and 1B show side-sectional views of a punching dilator 2 that incorporates two sections, a distal section 14 and a proximal section 24. The outer diameter of the distal section is smaller than that for the proximal section to enable expanding the opening through the host vessel wall in steps, which provides a better dilation effect as opposed to immediately expanding the opening through the host vessel wall to the large outer diameter. By expanding to a first diameter and creating a punch, or coring a section of tissue, prior to expanding to the second, larger diameter, the opening through the vessel wall has increased and tissue has been removed. Removing an aperture of tissue minimizes the stress on the vessel wall opening and decreasing the potential for splitting, which can occur from a dramatic instantaneous over-expansion of the tissue.

As shown in FIGS. 1A and 1B, distal section 14 has a tapered region, which provides a smooth transition from the guidewire or needle to the outer diameter of the distal section. A stylet 22 extending from the proximal section through the distal section defines a lumen 16 in which a guidewire or needle may be advanced and used as a guide over which punching dilator 2 may be advanced or retracted. Proximal section 24 also provides a tapered region to provide a smooth transition from the distal section to the outer diameter of the proximal section. In this embodiment, the proximal section contains a cutting element 10 designed to cut a section of tissue through the host vessel wall. The embodiment shown in FIGS. 1A and 1B incorporates a movable stylet 22 designed to axially move distal section 14 relative to proximal section 24 and execute the punching process. When distal section 14 is positioned forward, as shown in FIG. 1B, distal section 14 may be advanced through the host vessel wall such that the vessel wall fills the space between cutting element 10 and cutting surface or stop 12 of the distal section. Once the vessel wall is positioned against cutting surface 12, the cutting element is moved axially towards cutting surface 12 to punch an aperture through the host vessel wall. The cutting surface is preferably fabricated from a solid material that a cutting edge can slightly penetrate (e.g., acetal resin (such as DELRIN, Manufactured by E.I. du Pont de Nemours, Inc., Wilmington, Del.), FEP, PTFE, polyurethane, other polymer, etc.) so the cutting element can be assured to completely advance through the tissue and create an intact, well-defined core of tissue. Alternatively, cutting surface 12 may be fabricated from stainless steel, other alloy, or a polymer. The cutting element is preferably fabricated from an alloy (e.g., stainless steel) that may be sharpened to a blade. A deployment sheath (not shown) may be contained around the punching dilator and advanced through the opening once the punching dilator is positioned through the opening.

FIGS. 1C and 1D show an alternative punching dilator embodiment. In this embodiment, stylet 22 is offset from the center of proximal section 24. As such, stylet 22 of the punching dilator may be urged against the edge of the opening defined when distal section 14 is advanced through the host vessel wall. With the stylet against the pre-dilated opening edge, the punch may be accurately produced such that the cored aperture extends completely around the pre-dilated opening thus removing any discontinuities (e.g., splits) produced when advancing the distal section through the opening. When the stylet is positioned in the middle of the proximal section (as shown in FIGS. 1A and 1B) the punching dilator must be positioned with the stylet at the middle of the pre-dilated opening while punching the aperture of tissue. Otherwise, the punched aperture will not be completely round and will be more susceptible to splitting due to over-expansion. For cases where the punched aperture diameter greatly exceeds the pre-dilated opening produced when advancing the distal section through the host vessel wall, the location of the stylet is irrelevant. However, for those cases where the pre-dilated opening has approximately the same length as the diameter of the punched opening, orientation of the stylet relative to the dilated opening is essential to forming a complete punch of tissue.

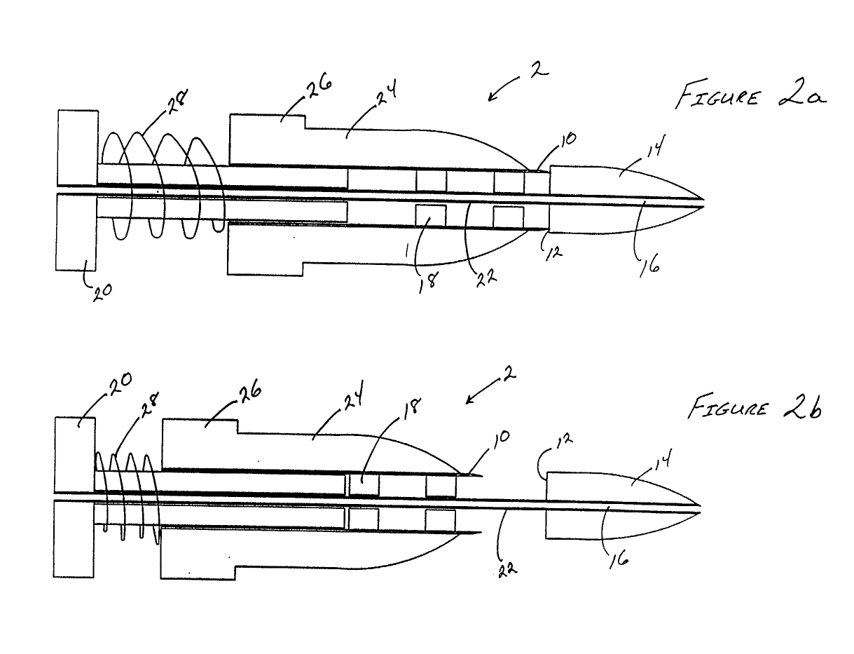

FIGS. 2A and 2B show a the punching dilator of FIGS. 1A and 1B with a spring 28 located between proximal section handle 26 and distal section handle 20 to ensure cutting element 10 remains against distal cutting surface 12 once the core of tissue has been created. This ensures punching dilator 2 will retain the section of tissue punched from the host vessel wall while the punching dilator is manipulated.

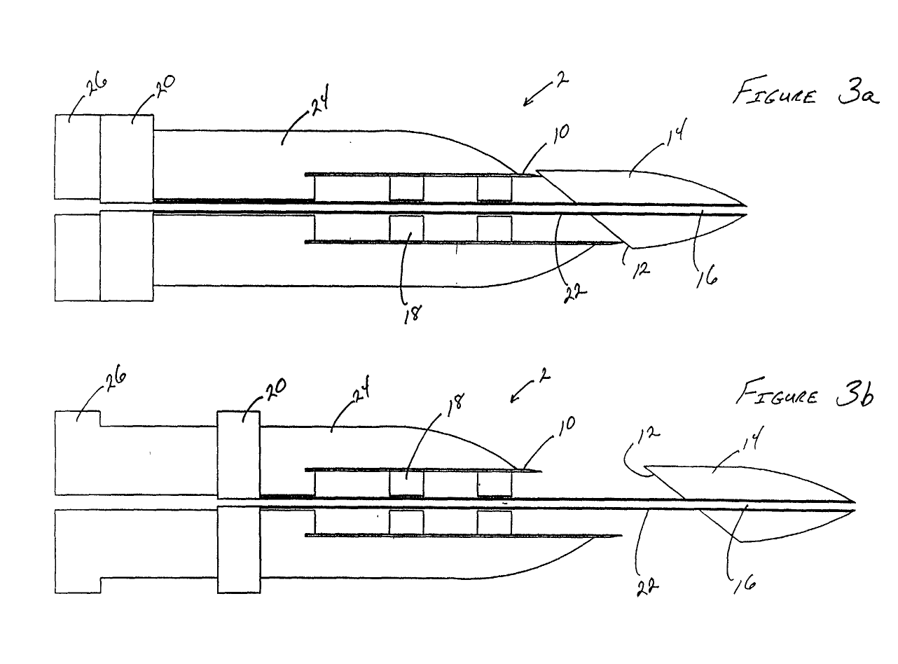

FIGS. 3A and 3B show an alternative punching dilator 2 that forms an oval aperture through the host vessel wall. By angling cutting element 10 and cutting surface 12, the punching dilator may be positioned at an angle relative to the host vessel wall. The angle determines the dimensions of the aperture. At about 90 degrees (characteristic of the punching dilator shown in FIGS. 1A and 1B), a circular aperture is created. At about 45 degrees (characteristic of the punching dilator shown in FIGS. 3A and 3B), an elliptical aperture is created with a length approximately equal to the square root of the width divided by two, Other angles and corresponding relationships between the length and width of the elliptical aperture may be utilized.

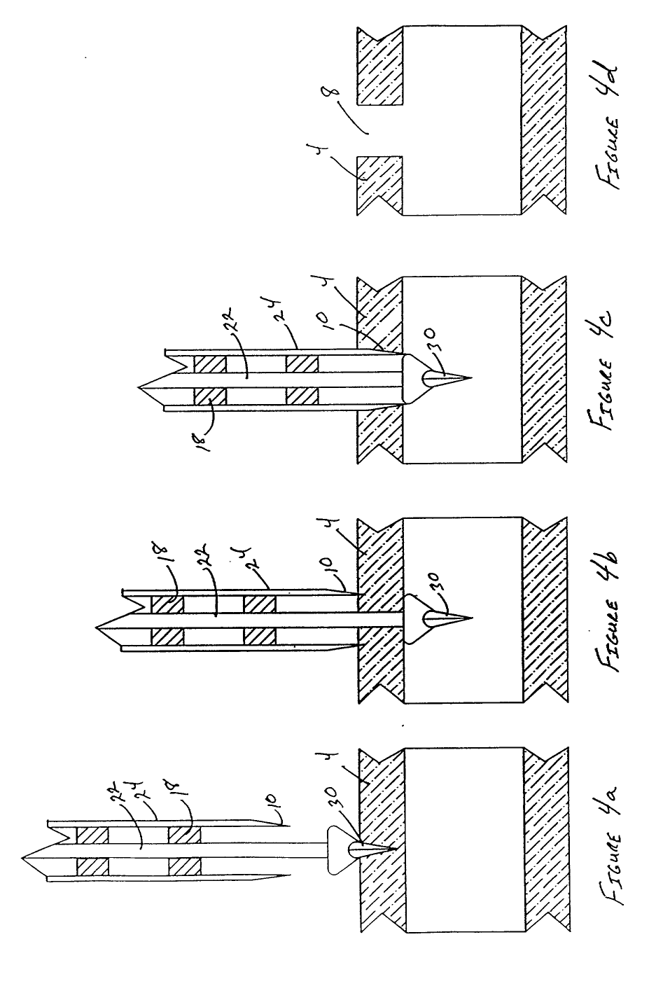

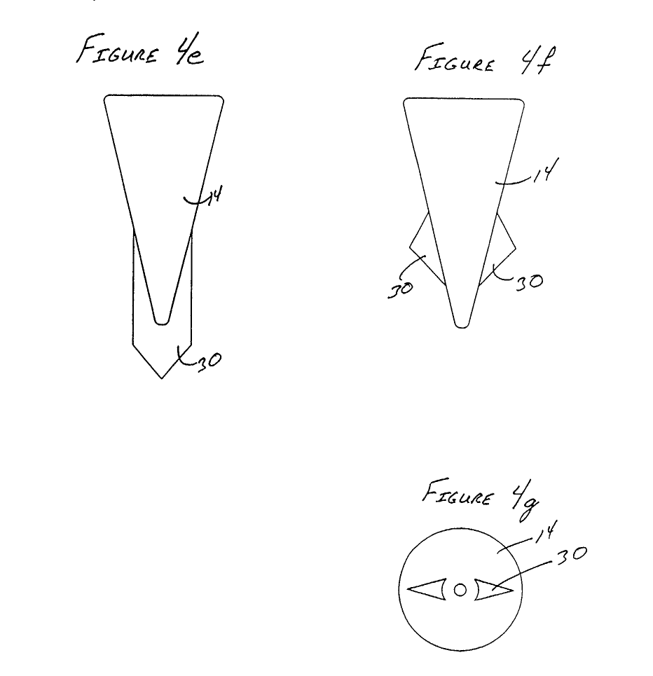

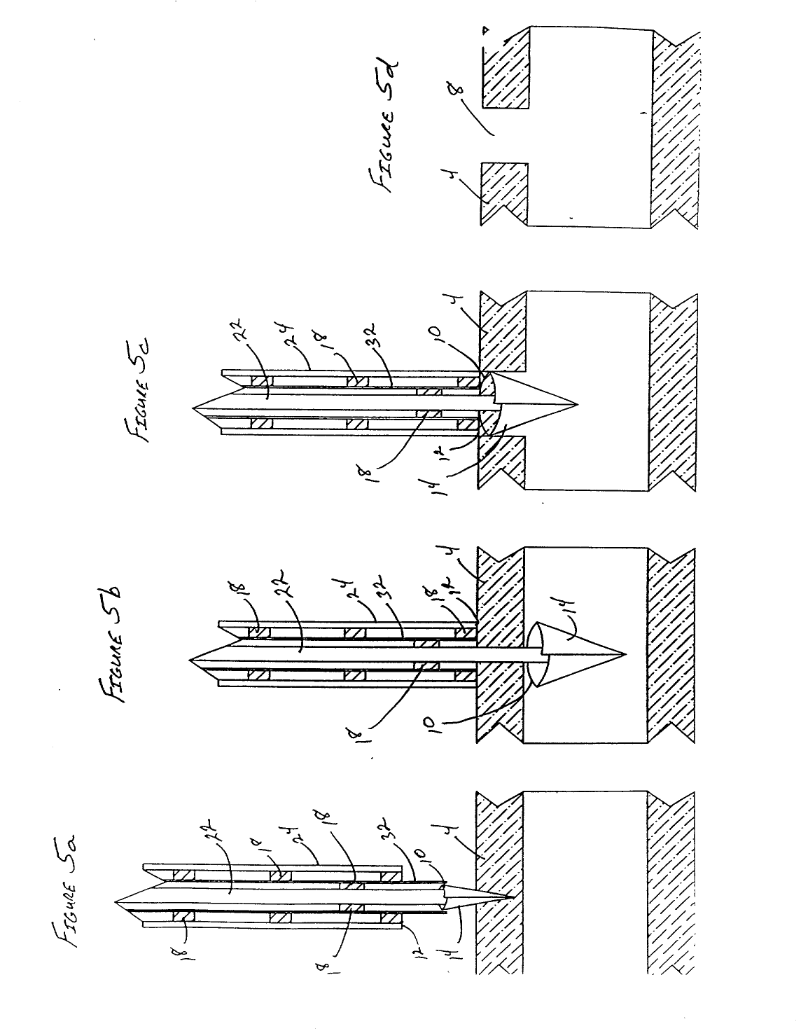

FIGS. 4A to 4D show the process of coring an opening through the host vessel wall using a punching dilator embodiment that incorporates a cutting or incising mechanism at the distal end. As opposed to creating the initial cut or puncture through the host vessel wall with a separate scalpel or needle, and using a guidewire to provide a conduit through the host vessel wall, a cutting mechanism 30 is disposed on the end of the distal section of the punching dilator. Once the initial incision is created, the distal end is inserted through the pre-dilated opening and the cutting surface is positioned against the interior surface of the host vessel wall, as shown in FIG. 4B. Once positioned, the cutting element of the proximal section is advanced relative to the cutting surface thereby punching an opening 8 through the host vessel wall, as shown in FIGS. 4C and 4D. A variety of cutting mechanisms may be utilized. FIG. 4E shown a cutting mechanism 30 emanating from the end of distal section 14. FIGS. 4F and 4G show an alternative cutting mechanism 30, which is advanced over a guidewire as previously discussed. The cutting mechanism consists of opposing cutting elements 30 disposed on opposite sides of the distal section 14 and offset from the end of the distal section. As such, the cutting elements follow the end of distal section 14 through the opening in the host vessel wall (over a guidewire or needle) and create an incision when the distal section is advanced through the host vessel wall past the cutting elements 30. This embodiment helps prevent advancing the cutting mechanism against the posterior wall of the host vessel which could pose complications if the punching dilator cut into the intima or completely through the posterior wall of the host vessel.

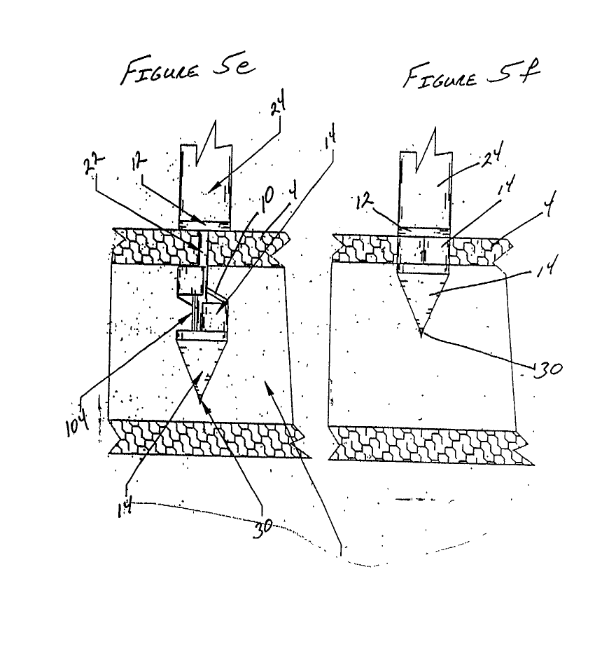

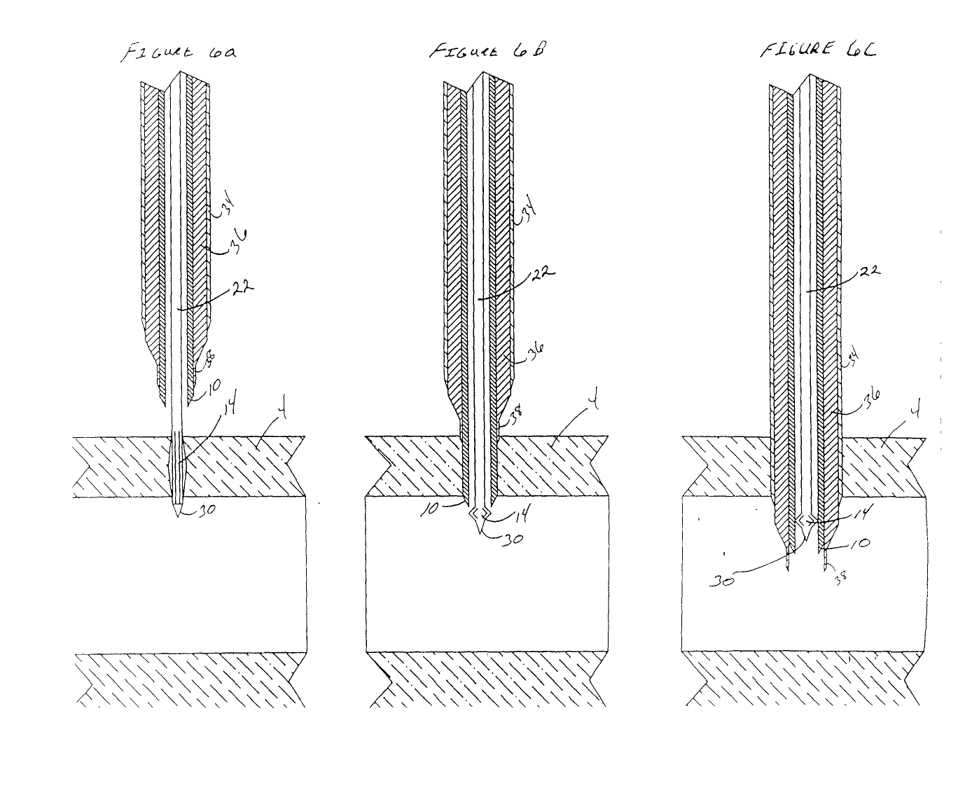

FIGS. 5A to 5D show an alternative punching dilator used to create an opening through the host vessel wall. This punching dilator incorporates a compressible, expandable distal section 14. As shown in FIG. 5A, distal section 14 is formed as a cone with opposing sides overlapping. As such, distal section 14 may be compressed into a small diameter by wrapping opposing sides to decrease the outer diameter. As shown in FIG. 5A, a constraining tube 32 maintains the compressed orientation of the distal section while advancing through the host vessel wall. The distal section forms a pointed distal end capable of puncturing tissue. As shown in FIG. 5B, constraining tube 32 is retracted relative to distal section 14 to allow the distal section to expand towards its preformed configuration. Distal section 14 incorporates a cutting element 10 at its proximal end such that once distal section 14 is advanced through the host vessel wall, is allowed to expand into the enlarged diameter orientation, and is withdrawn towards cutting surface 12 (located at the end of the proximal section 24), a core of tissue from the host vessel wall is removed thereby defining aperture 8.

FIGS. 5E and 5F show an alternative punching dilator that incorporates a compressible, expandable distal section. This distal section incorporates a coiled cutting element 10 that may be stretched into a reduced diameter profile for advancing through the host vessel wall. Once inside the host vessel, the distal section is allowed to (or urged to) return towards its enlarged diameter orientation and cutting element 10 of the distal section is retracted against cutting surface 12 of the proximal section to produce a punch in the host vessel wall.

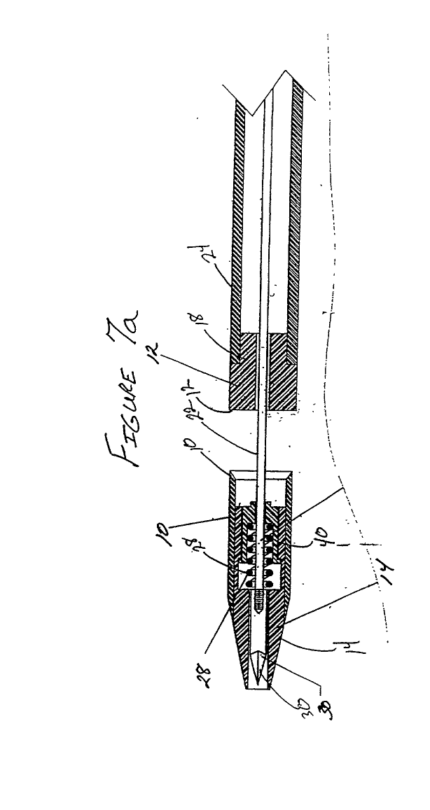

FIGS. 6A to 6C show an alternative punching dilator. This punching dilator incorporates a compressible, expandable distal section which consists of a slotted tubing that is preformed into a cutting surface, in the enlarged diameter configuration.

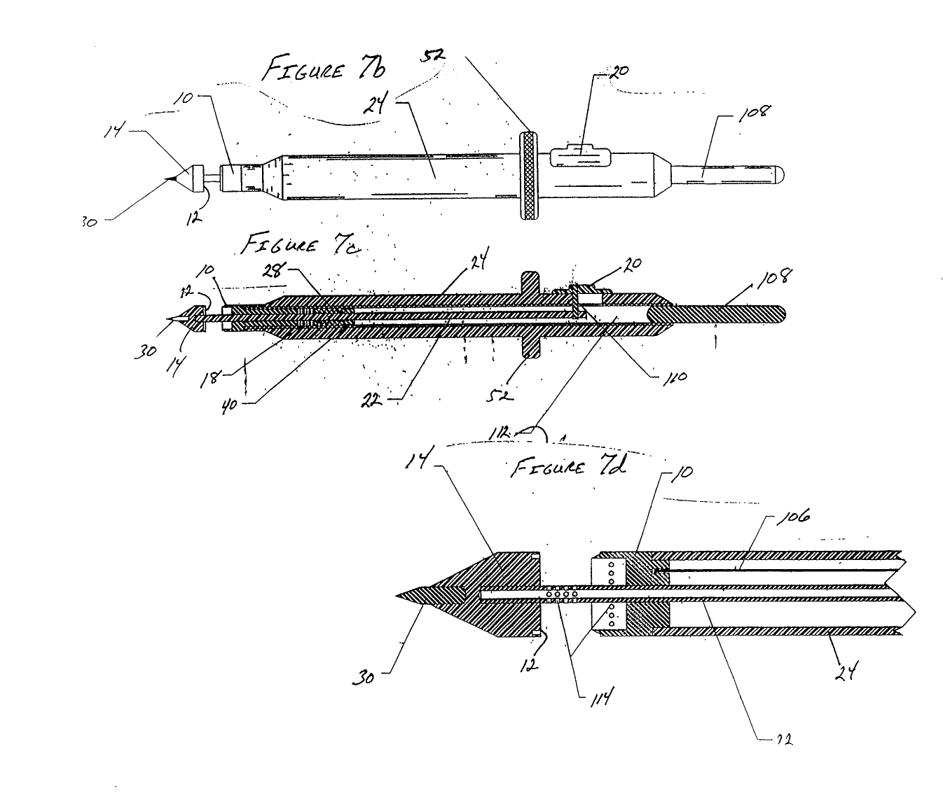

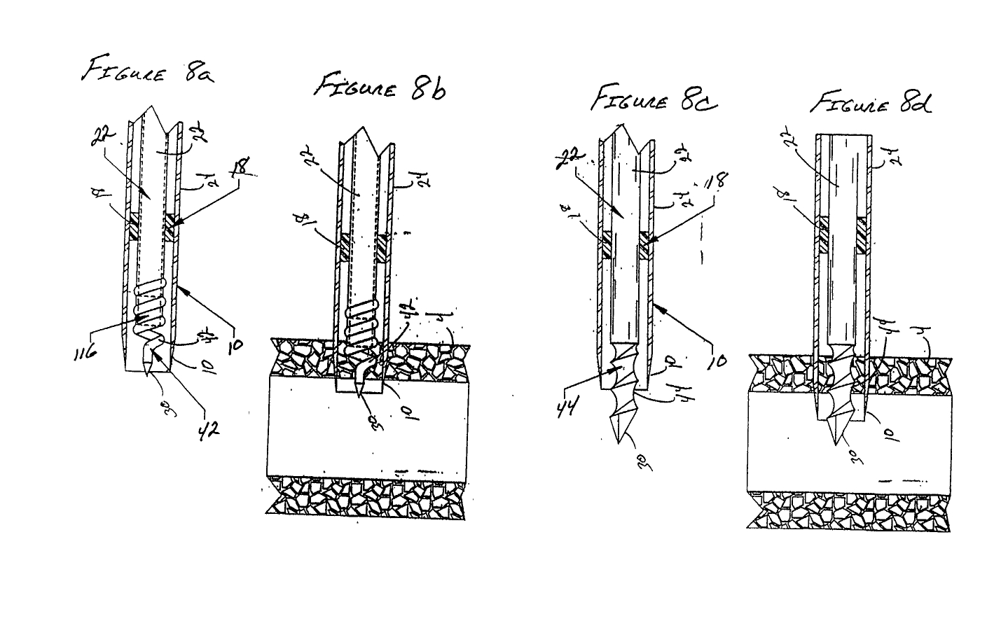

For a number of the previously described punching dilator embodiments that incorporate a cutting mechanism at the distal end to produce the initial opening through the host vessel wall, the cutting mechanism is exposed throughout the punching process. As such, over-advancing or excess manipulation may damage the host vessel wall away from the section of host vessel wall to be cored. FIG. 7A shows the distal end of a punching dilator that incorporates a shielding mechanism to cover the distal cutting mechanism 30. A stylet 22 is attached to cutting mechanism 30 and moves axially relative to proximal section 24. The cutting mechanism 30 may be a needle or scalpel blade, as previously described. A bearing 18 controls the radial position of stylet 22 relative to the proximal section. Also attached to stylet 22 is a spring housing 40 which provides a surface from which the spring 28 exerts force. Distal section 14 retracts axially toward the proximal section when the end of the distal section is pressed against tissue. This exposes cutting mechanism 30 so as to cut an initial opening in the host vessel wall. Once distal section 14 is completely advanced through the host vessel wall, the distal section 14 springs forward shielding cutting mechanism 30. Distal section 14 also incorporates a cutting element 10 at the opposite end to punch tissue and create the aperture. The end of the proximal section 24 acts as a cutting surface 12 onto which cutting element 10 cores a section of tissue. Once distal section 14 is completely engaged against cutting surface 12 of the proximal section, distal section 14 is unable to retract thereby ensuring the cutting mechanism 30 is shielded. Once the punch is created, the punching dilator is further advanced through the opening so as to position the access sheath. By shielding the cutting mechanism 30, during access sheath positioning, this punching dilator embodiment prevents damaging the posterior region of the host vessel wall while advancing or otherwise manipulating the punching dilator.

FIGS. 7B to 7D show punching dilator embodiments that incorporate the ability to cauterize the host vessel wall while coring an opening through the host vessel wall. An electrosurgical unit is coupled to proximal connector 108 using standard cabling. Proximal connector 108 is routed to cutting element 10 located on proximal section 24, in this embodiment (cutting element 10 may alternatively be located on distal section 14), using a signal wire 112 or 106. As cutting element 10 is advanced against cutting surface 12, the transmission of radiofrequency energy into the host vessel wall produces a cauterizing effect, which produces a well-defined opening through the host vessel wall. As shown in FIG. 7D, holes 114 may be incorporated in stylet 22 and/or cutting element 10 so that fluid may be injected to further localize the cauterizing effect and prevent coagulation of adjacent, unwanted tissue. In this embodiment, a fluid injection port is located at the handle of the punching dilator and is connected through the lumen of stylet 22 and the proximal section and to holes 114 so fluid may be injected into the region between cutting element 10 and cutting surface 12.

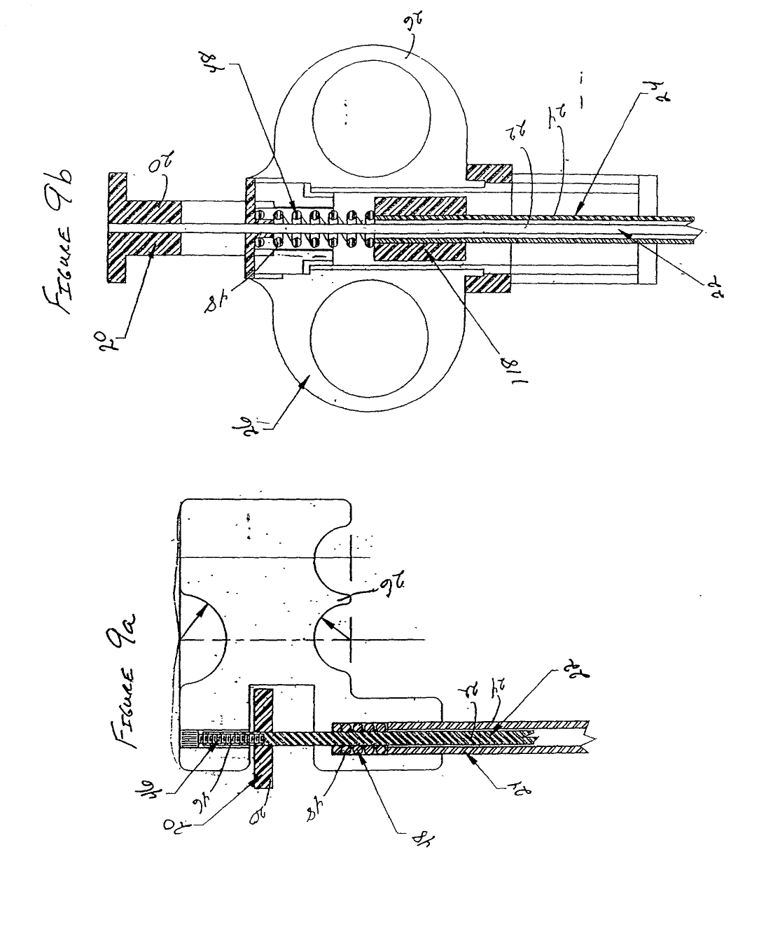

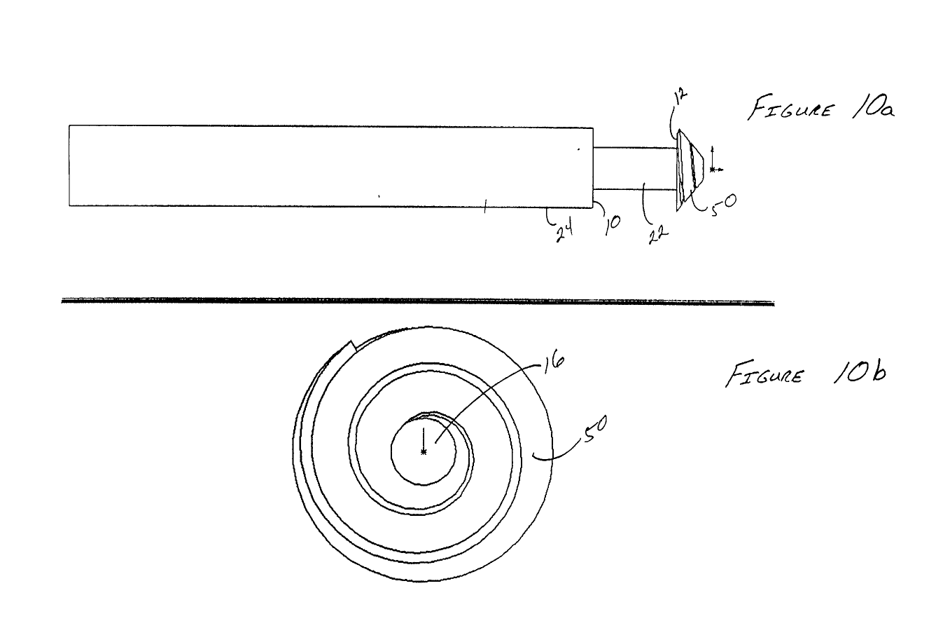

As opposed to producing an initial cut through the host vessel wall to position the distal section, a modification to the punching dilator capable of minimizing the splitting associated with pre-dilating the opening is to include a screw-in mechanism in the distal section of the punching dilator, as shown in FIGS. 8A and 8B. The screw-in punching dilator facilitates insertion of the distal section by invoking rotation of the distal section through the opening in the host vessel wall. A cutting element 30 is incorporated at the distal end of screw 42 to produce the initial cut through the host vessel wall. By rotating screw 42, the helical winds of the distal section are advanced into the host vessel wall at a controlled rate, preventing the splitting response associated with dramatic pre-dilation of the opening. Once screw 42 is positioned into the host vessel wall, it produces an anchor against which cutting element 10 may be advanced and used to punch a section of tissue. An alternative embodiment for the screw-in punching dilator is shown in FIGS. 8C and 8D. The distal section incorporates an auger bit with threads 44 (as opposed to helical winds as previously discussed) to provide the surface to screw the punching dilator into the host vessel wall. Once the threads are positioned, cutting element 10 is advanced thereby punching a section of tissue and defining the aperture to advance the proximal section of the punching dilator and the associated access sheath.

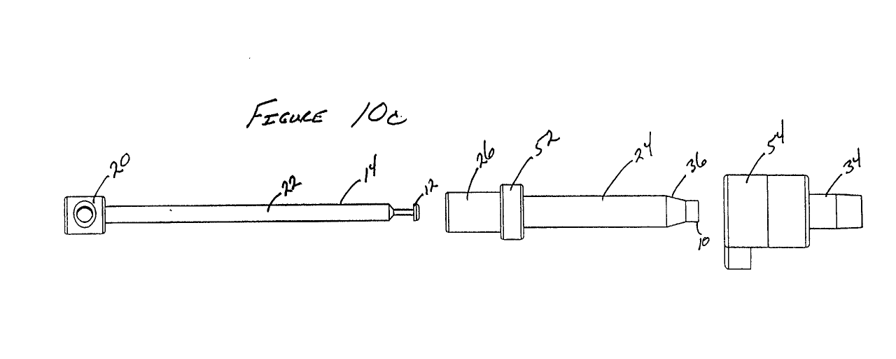

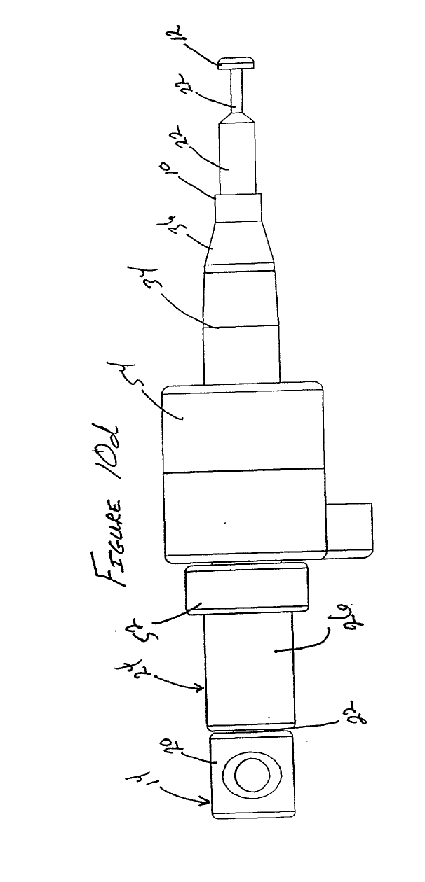

FIG. 9A shows a representative handle mechanism to controllably rotate the helical winds of the screw or threads of the auger bit, previously described. The handle incorporates a proximal handle member 26 and a distal handle member 20 designed to control the movement of proximal section 24 relative to the distal section. Proximal handle member 26 matches the palm of an operators hand and provides a surface from which to rotate the distal handle member 20. A proximal handle screw 46 connects distal handle member 20 to proximal handle member 26 and controls the rotation and advancement of stylet 22 upon rotation of distal handle member 20. The movement of stylet 22 in turn determines the position of the distal section which includes the screw-in mechanism.

FIG. 9B shows a representative handle mechanism to axially move the distal section relative to the proximal section for those punching dilator embodiments that punch a section of tissue by urging cutting element 10 (attached to one of the distal section or proximal section) against cutting surface 12 (incorporated on the other of the distal section or proximal section). Distal handle member 20 is attached to the stylet 22 and axially moves relative to proximal handle member 26, which is attached to proximal section 24. A spring 48 is disposed between distal handle member 20 and a proximal stop 118 that is secured to proximal handle member 26. The spring urges distal handle member 20 either away from proximal handle member 26 to maintain the distal section against the proximal section when in the relaxed configuration or towards proximal handle member 26 to urge the distal section away from the proximal section in the relaxed configuration.

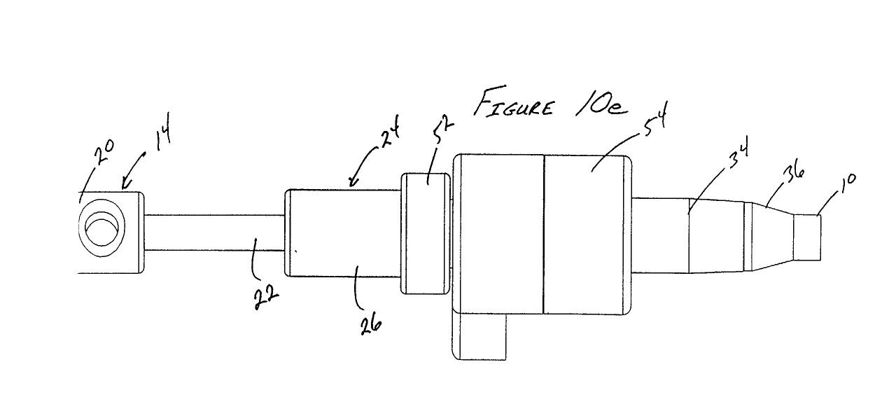

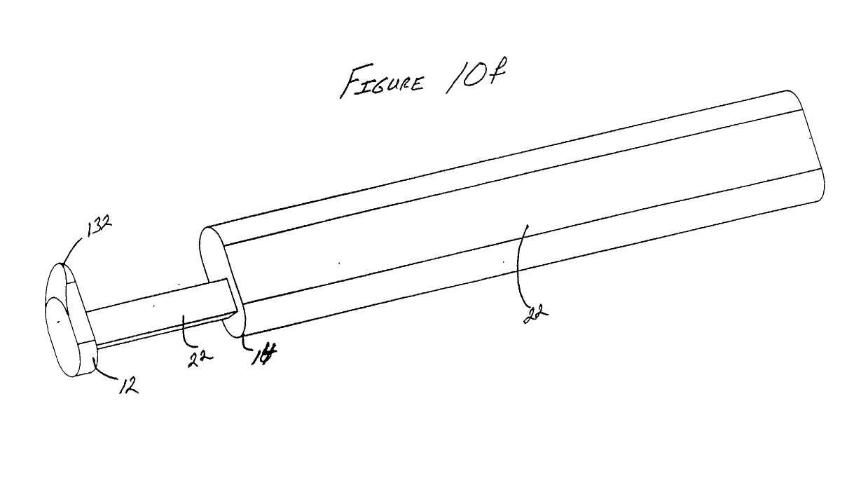

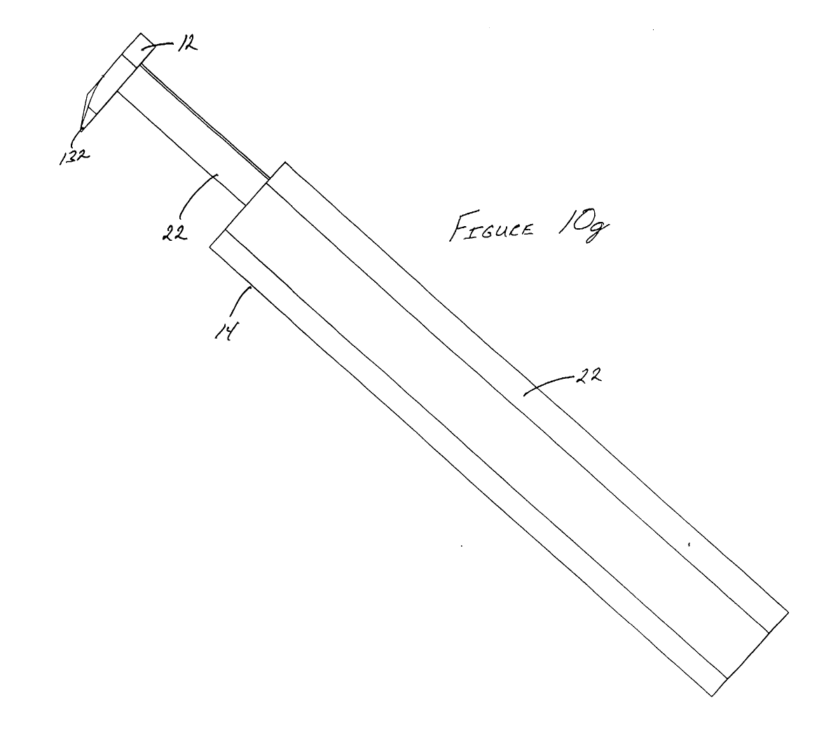

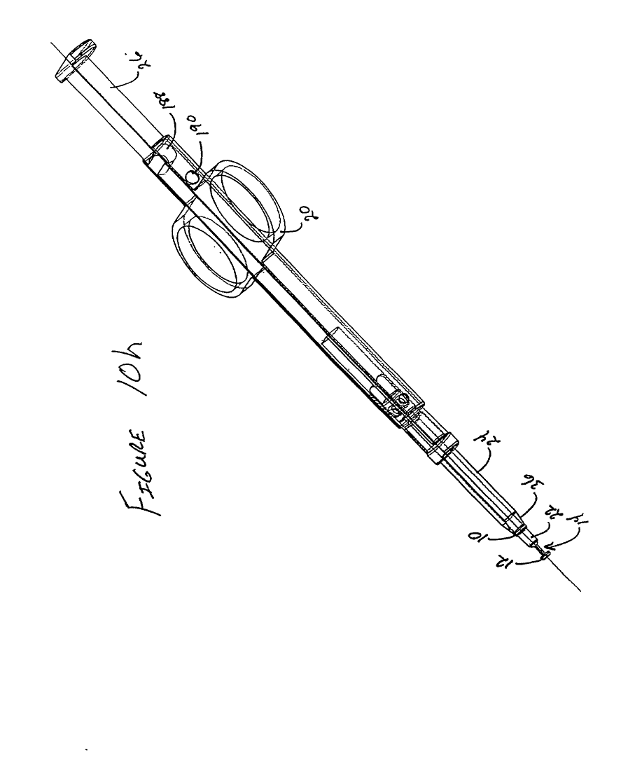

FIGS. 10A and 10B show an alternative screw-in punching dilator. The distal section incorporates a tapered screw mechanism 50, which is advanced through the host vessel wall upon rotation of the distal section. The distal section also includes a lumen 16 that is routed through stylet 22 and provides a conduit to advance a guidewire or needle and provide a guide to controllably advance the distal section through the host vessel wall. Proximal section 24 includes a cutting element 10 which creates a punch of tissue when urged against cutting surface 12 located on the proximal end of the distal section.

FIGS. 10C to 10E show a punching dilator that incorporates a scissors mechanism to cut a core of tissue. The punching dilator incorporates a distal section 14 that fits within proximal section 24. The distal section incorporates a thin cutting surface 12 at the distal end. The outer diameter of stylet 22 matches the inner diameter of proximal section 24 such that the distal section moves axially relative to the proximal section but restricts excess, unwanted radial movement. A tapered section 36 transitions proximal section 24 from the outer diameter of cutting element 10 to the outer diameter of the proximal section. Ideally, this tapered section 36 extends at an angle less than or equal to approximately ten degrees from the cutting element providing a smooth transition to insert the punching dilator and prevent abrupt over-expansion, which may cause the host vessel wall to split radially. An access sheath has a hub 54 and tubing 34 designed to fit around the exterior surface of proximal section 24 and permit axial movement along the proximal section. Once assembled as shown in FIG. 10D, the punching dilator is prepared to create a core of tissue through the host vessel wall. An incision is created in the host vessel wall and cutting surface 12 is slid through the incision and positioned into contact with the interior surface of the host vessel wall. Once positioned, the proximal section 24 is moved relative to the distal section to cause the cutting element 10 to pass over the outer diameter of the cutting surface 12 causing tissue within the inner diameter of the cutting element to be cut away from the host vessel wall, as shown in FIG. 10E. This scissors action produces a well-defined punch and urges the cutting element through the aperture defined by the punching dilator. Once the punch is created, the punching dilator is advanced until access sheath tubing 34 resides through the aperture and provides a conduit into the interior of the host vessel. The punching dilator is removed, leaving the access sheath through the aperture in the host vessel wall.

FIGS. 10F and 10G show an alternative distal section embodiment for the scissors punching dilator described above. This distal section 14 incorporates cutting mechanism 132 on one side of cutting surface 12 to produce an incision through the host vessel wall and slide the cutting surface through the incision without having to use a separate scalpel or other blade. Cutting mechanism 132 may consists of sharpening the thin cutting surface 12 into a sharp blade. This distal section 14 also enables creating an elliptical punch having a desired width and length, as determined by the cross-section of cutting surface 12 and the cutting element (not shown). Of course, the proximal section (especially the cutting element) would ideally match the distal section to effect the desired punch.

FIG. 10H shows an assembled punching dilator that incorporates a scissors mechanism to shear tissue and cut an opening through the host vessel wall. As previously described, distal section 14 incorporates stylet 22 and cutting surface 12. The distal section is attached to distal section handle 20, which incorporates eyelets through which the operator may position fingers to hold the punching dilator. Proximal section 24 incorporates the cutting element 10, which punches an opening through the host vessel wall as it is advanced over cutting surface 12 of the distal section. The proximal section has a tapered region 36 that extends from the outer diameter of cutting element 10 to the outer diameter of proximal section 24, over which the access sheath is positioned for deployment through the opening in the host vessel wall. The proximal section is attached to a proximal section handle member 26 which may be advanced axially relative to distal section handle member 20. The distal section handle member incorporates a locking hole 190 through which a spring loaded locking ball 188 (located on the proximal section handle member) extends when locking ball 188 enters the notch defined by locking hole 190. This locks the position of the proximal section relative to the distal section once cutting element 10 is advanced over cutting surface 12 and a section of tissue is punched. As such the cut tissue is restrained between the proximal section and distal section eliminating the possibility that the cut tissue may become an embolus. To enable reusing the punching dilator, locking ball 188 is manually pressed past the confines of the locking hole 190 so the proximal section may be retracted relative to the distal section. The cut tissue may then be removed from around the distal section and the punching dilator used to create another opening through the host vessel wall.

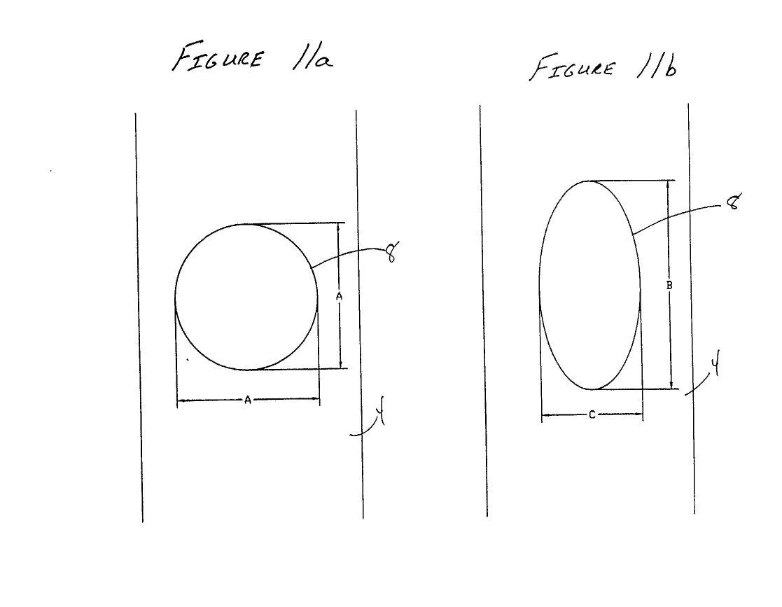

FIGS. 11A and 1B show potential punch cross-sections that are obtainable with the punching dilators described above. Other cross-sectional geometries may be utilized as desired. The advantage of creating an elliptical punch stems from the ability to decrease the surface area of the punch and increase the resistance of the opening to splitting. During experimental studies, elliptical punches oriented axially relative to the host vessel may be expanded to significantly larger diameters without splitting than circular punches having the same cross-sectional area, or elliptical punches oriented radially relative to the host vessel. As such, hemostasis was improved when using elliptical punching dilators oriented axially relative to the host vessel.

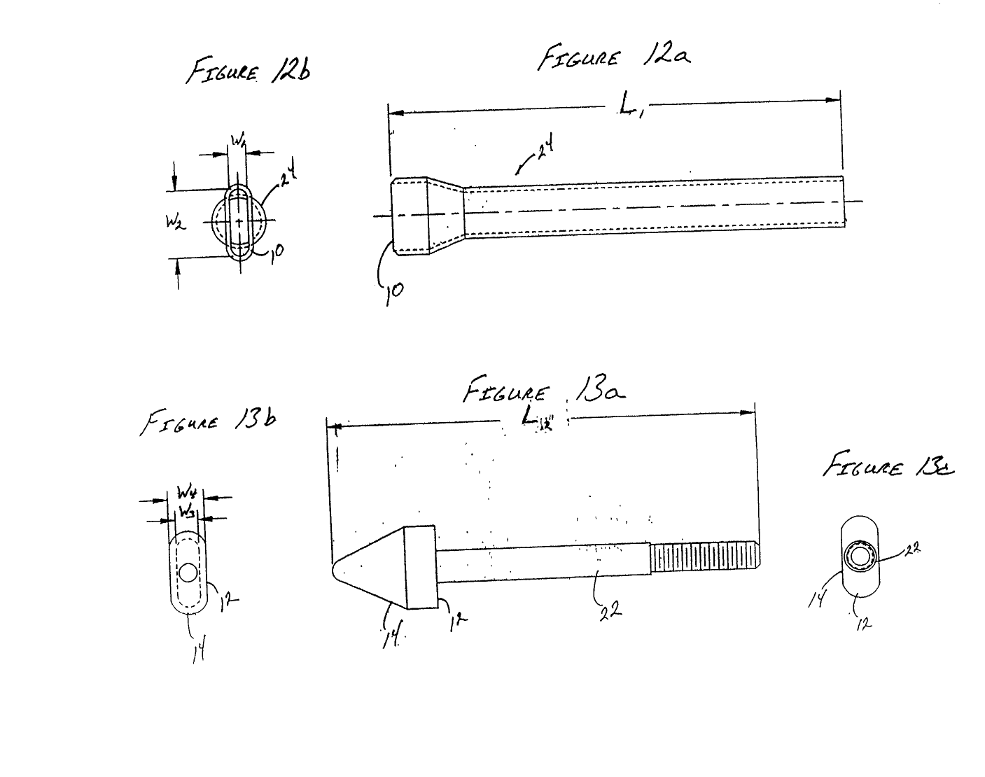

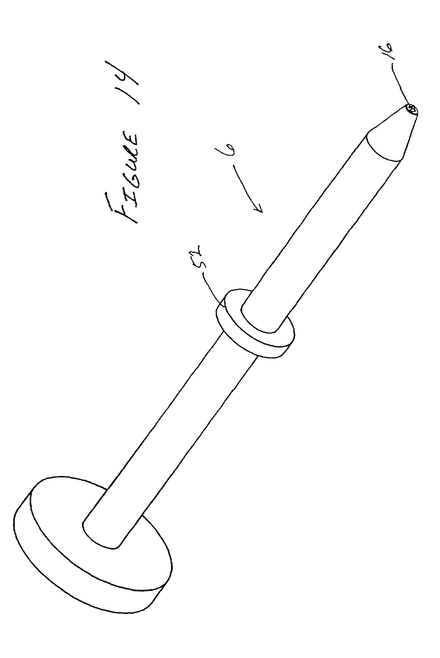

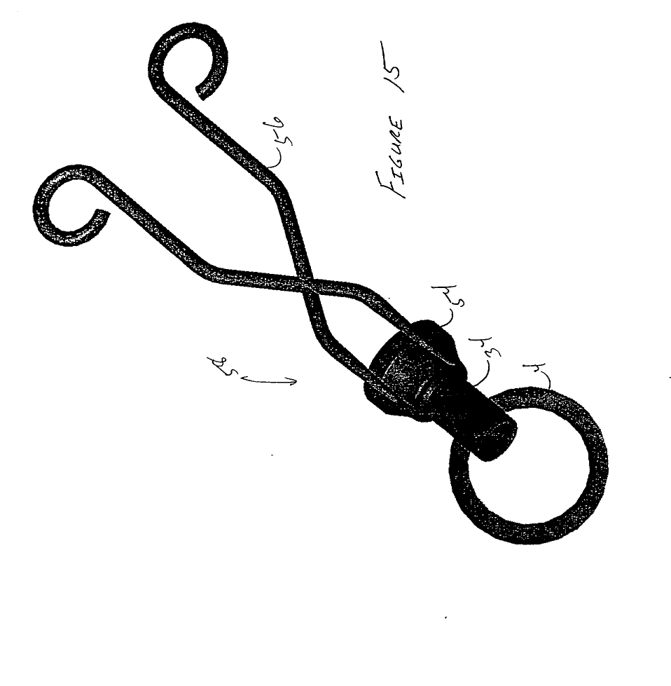

FIGS. 12A and 12B show a proximal section 24 to a scissors punching dilator, which incorporates an elliptical cutting element 10. The corresponding distal section 14 is shown in FIGS. 13A to 13C. The main body of the proximal section and stylet 22 may be circular as shown in FIGS. 12A and 12B, and FIGS. 13A to 13C; alternatively, the main body and stylet 22 may be fabricated with an elliptical cross-section or other geometry. By utilizing an elliptical cross-section or other locking geometry on the stylet and main body, the orientation of cutting surface 12 relative to cutting element 10 is maintained to ensure the cutting element 10 moves over cutting surface 12 and produces a well-defined punch of tissue.

Several of the punching dilator embodiments described above eliminate the need for using a guidewire to insert the distal section through the initial opening into the host vessel wall.

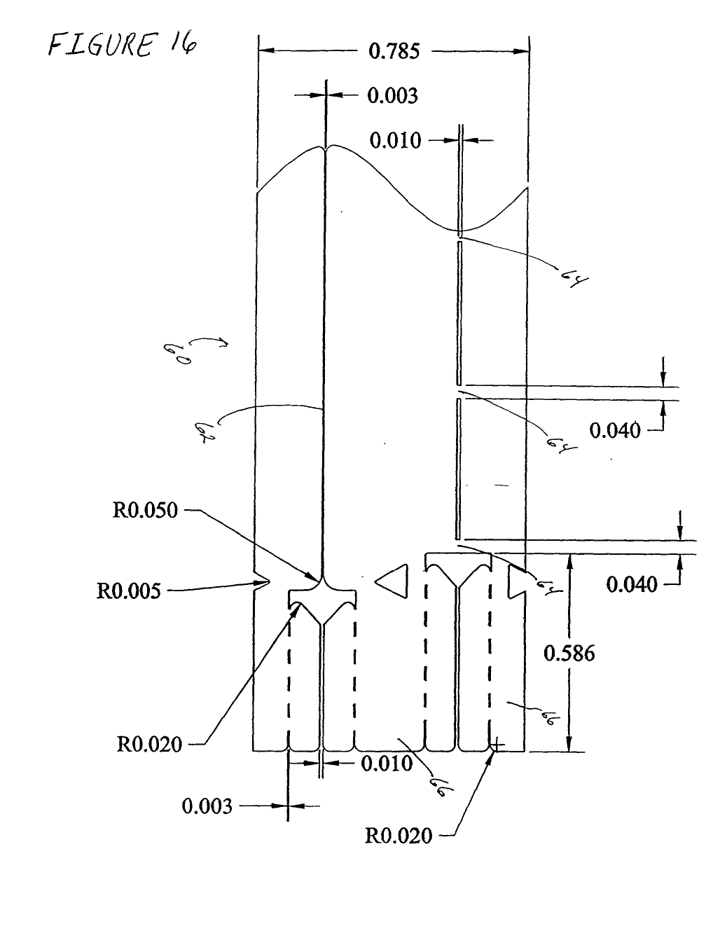

Whether conventional dilators are used or punching dilators are used, a stop 52 needs to be used to position the access sheath during deployment, as shown in FIG. 14. The stop helps locate the access sheath such that the access sheath tubing resides at the proximal end of the taper to ensure a smooth transition to the access sheath tubing.

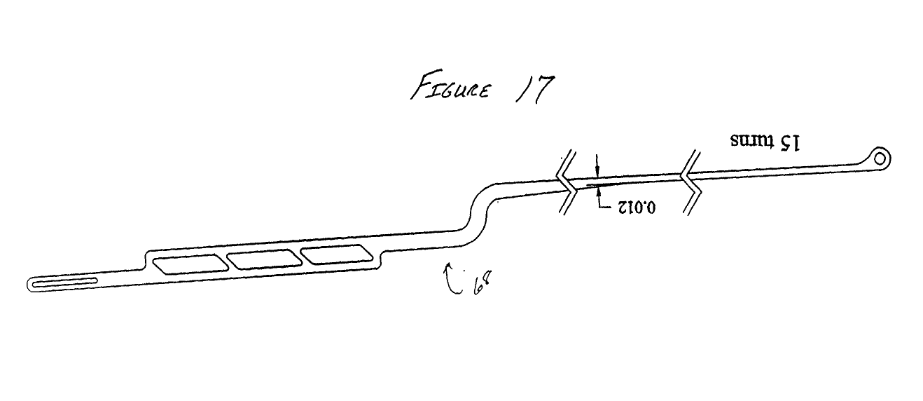

FIG. 15 shows an improvement to the splittable access sheath, which permits remote separation of the splittable deployment sheath into two separate but remotely attached components. This facilitates removal of the deployment sheath from the side of the bypass graft. After the bypass graft is attached at the anastomosis site using the end-side fittings as previously discussed, the clamp mechanism of the access sheath is actuated to separate the two halves of the access sheath. This access sheath embodiment has a main tubing 34 with a hub 54 incorporating an integrated hemostatic valve, opposing grooves, splits, or perforations to permit separation of the sheath halves, and extensions 56 that form a clamp mechanism to facilitate remote manipulation of the access sheath halves. Each half of hub 54 is connected to opposing extensions 56 that intersect at a pivot and extend to handles. Clamp 56 locks the two halves together during deployment of the access sheath through the host vessel wall or delivery of the end-side fitting through the access sheath. As the handles are unlocked and separated, the halves of the access sheath are urged apart providing a remote mechanism to split the access sheath.

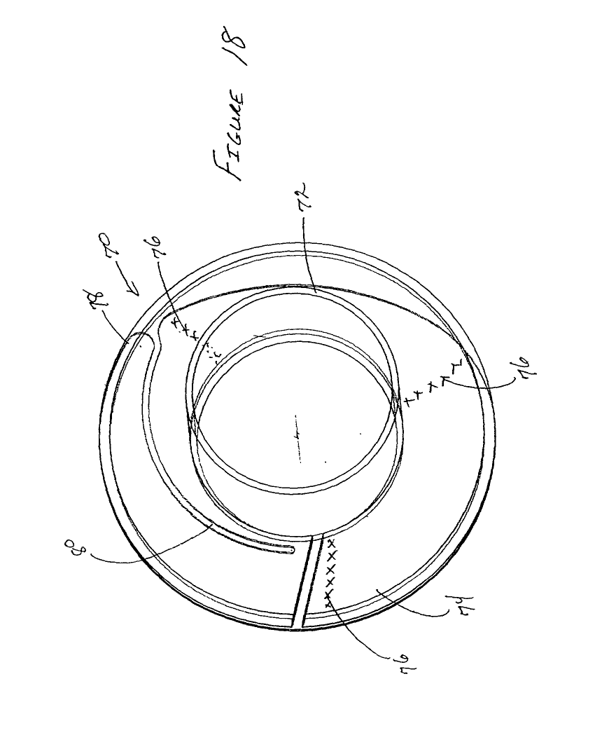

FIG. 16 shows a flattened profile of a splittable loading sheath 60. The loading sheath is fabricated from a tube that is laser cut into the desired pattern. The loading sheath incorporates a bevel at the distal end, two handle regions 66 to enable separation of the loading sheath, links 64 to permit movement of the two sides of the loading sheath, and a gap 62 from which the opposite sides of the loading sheath are separated. Once the end-side fitting and bypass graft are positioned through the loading sheath and secured to the host vessel wall, the access sheath is removed. After splitting and removing the access sheath, the loading sheath must be removed from around the bypass graft. To enable remote separation of the loading sheath, opposing handle halves 66 are squeezed together causing the halves of the loading sheath to rotate around links 64 and define a gap 62 with sufficient width to remove the loading sheath from around the bypass graft.

End-Side Fitting Improvements

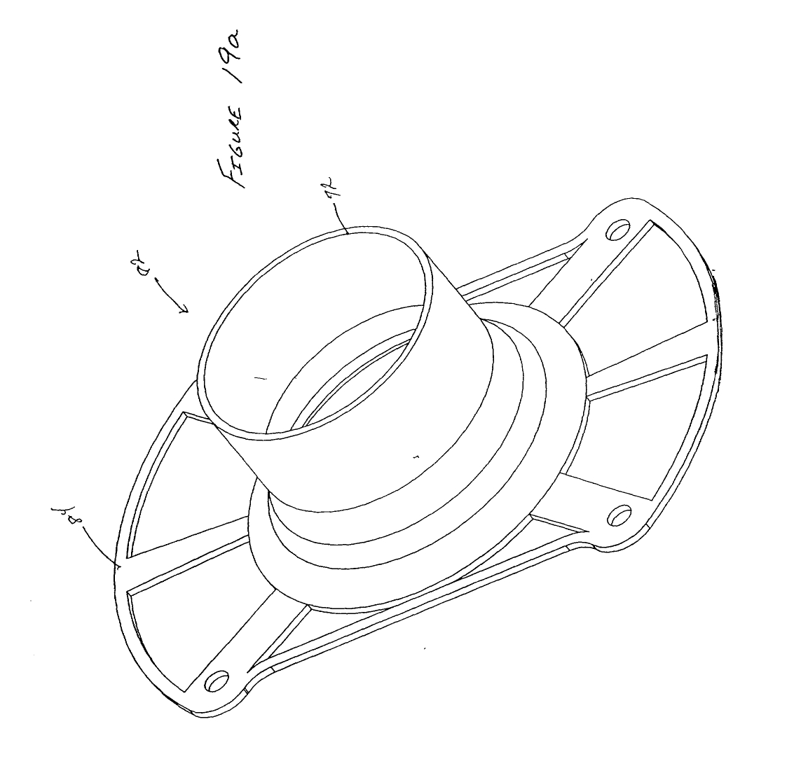

As discussed in co-pending U.S. patent application Ser. No. 09/329,503, a strain relief attached to the end-side fitting and extending a desired length along the bypass graft prevents kinking of the bypass graft when it emanates at acute angles from the anastomosis and prevents acute over-expansion of the bypass graft. FIG. 17 shows a flattened profile of a strain relief used to reinforce the bypass graft. Strain relief 68 incorporates a stiff distal end to compress the bypass graft against the stem of the end-side fitting and secure the bypass graft to the end-side fitting. The proximal end of strain relief 68 is tapered in stiffness to provide a smooth transition in compliance of the bypass graft from the anastomosis to the main body of the bypass graft.

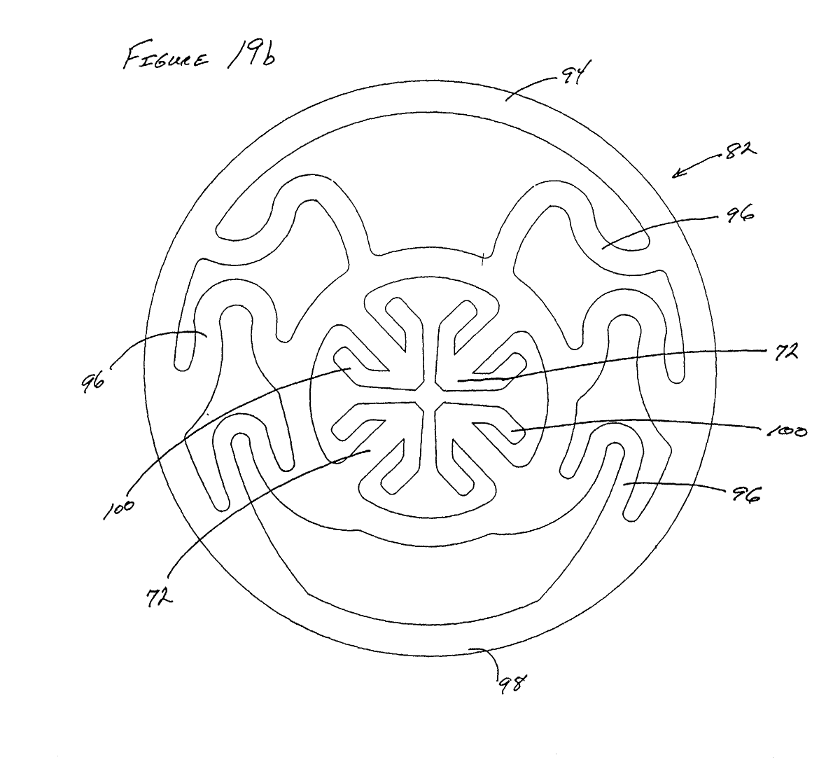

As discussed in co-pending U.S. patent application Ser. No. 09/329,503 and co-pending U.S. Provisional Patent Application Serial No. 60/111,948, entitled “Bypass Graft Positioning and Securing System”, to Houser et al., filed Dec. 11, 1998, each of which is incorporated herein by reference, end-side fitting embodiments having specific characteristics may be inserted through a small puncture without the need for an access sheath. FIG. 18 shows a screw-in, dilating end-side fitting 70 that meets these requirements. The screw-in, dilating fitting 70 incorporates a base or stem 72, a leading edge 78, and a slot 80 through the leading edge to cause the fitting to advance through a small opening in the host vessel wall as the fitting is rotated. A skirt or covering 74 is attached to the screw-in, dilating fitting at specific locations 76 around the fitting. Skirt or covering 74 maintains hemostasis at the anastomosis site even if splitting occurs during the deployment of the fitting through the host vessel wall. The screw-in, dilating end-side fitting may incorporate a feature that enables following a guiding mechanism (e.g. guidewire, needle, or small dilator) that directs the fitting into the host vessel interior while the screw-in, dilating fitting defines an opening through a host vessel wall.

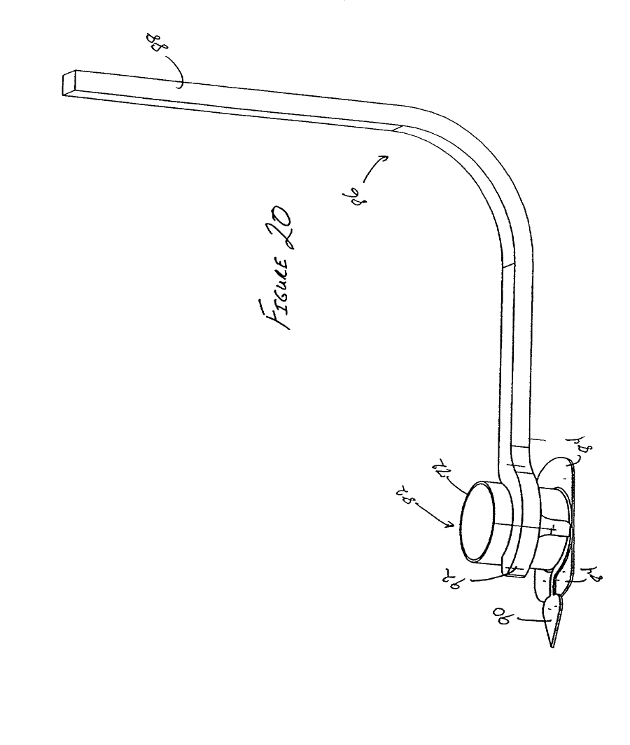

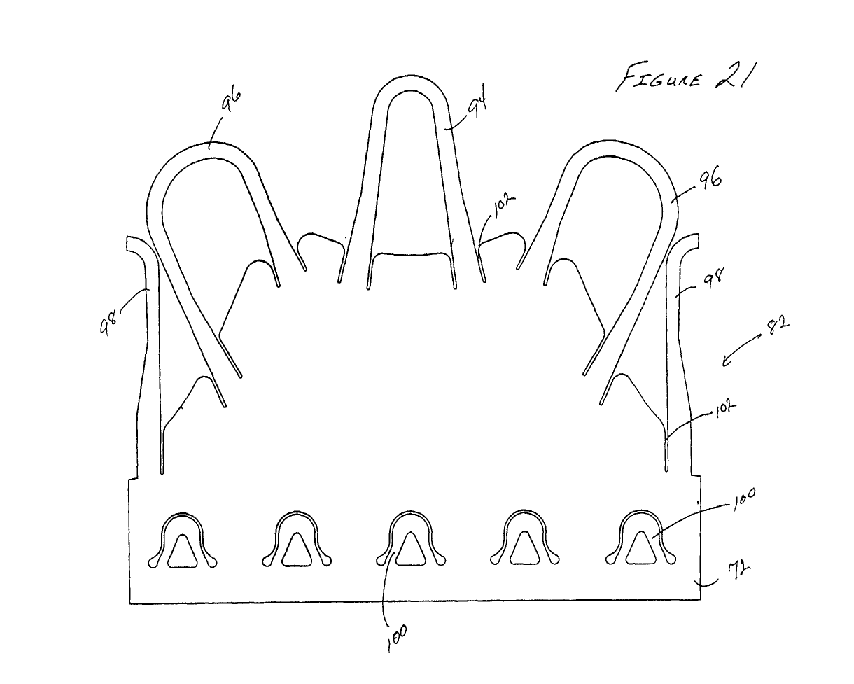

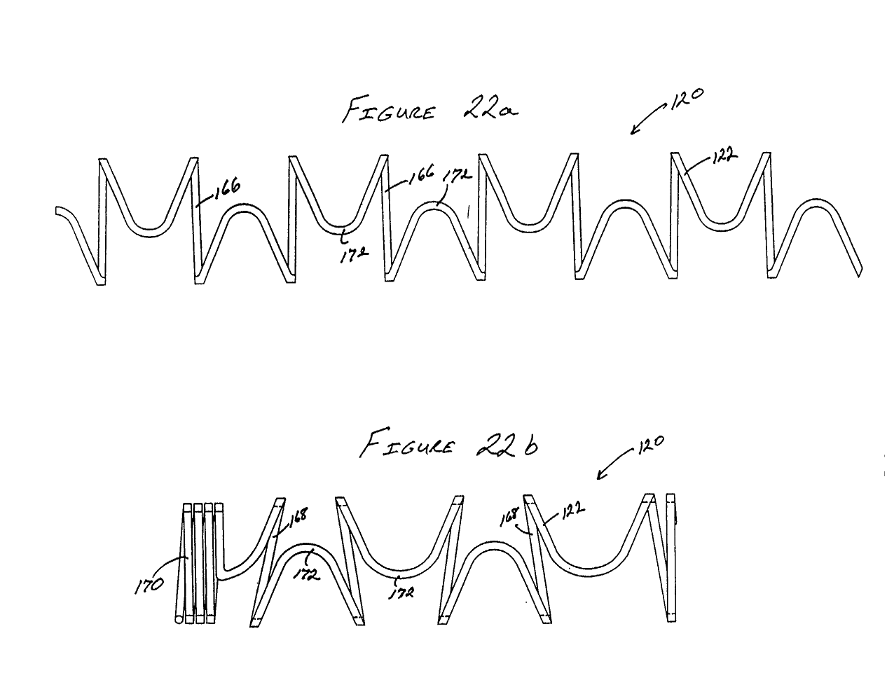

FIG. 19A shows another sheathless end-side fitting. This sheathless end-side fitting does not need to be rotated to be deployed through the host vessel wall. The sheathless end-side fitting 82 has a dilating end 84 that expands the opening through the host vessel wall, especially if the opening is an incision or elliptical punch. Once the front end of dilating fitting 82 is positioned within the host vessel, the fitting is further advanced until the rear end of the dilating fitting 82 is contained within the host vessel. Base or stem 72 of the fitting is then positioned within the opening of the host vessel wall to produce the anastomosis. A deployment device 86, as shown in FIG. 20, may incorporate an incision mechanism 90 to cut and expand the incision thereby providing a smooth transition to front end 84 of dilating fitting 82.

FIG. 19B shows a flattened profile of another sheathless end-side fitting 82 embodiment designed to be advanced through an incision or punch without the need for an access sheath. This sheathless end-side fitting embodiment is preferably fabricated from a flat sheet of superelastic material. The pattern of petals 96, stem 72, and tabs 100 is created using chemical etching, laser cutting, or other process. Once the pattern is cut, stem 72 and tabs 100 are stressed and thermally formed into the desired configuration. Petals 96 are also thermally formed to match the geometry of the host vessel. For large vessels the petals 96 extend away from the stem at an approximate 90 degree angle. For small vessels, the petals 96 are curved away from the stem such that the radius of curvature, in the thermally formed, relaxed orientation, approximates the radius of curvature for the host vessel. Petals 96 of this sheathless end-side fitting 82 define a leading petal 94 and a trailing petal 98. The leading and trailing petals may be deflected into a relatively tight radius of curvature upon deflecting petals 96 away from the stem and towards the central axis of the fitting. This way, the petals form a reduced diameter profile capable of being inserted through an incision or punch without the need for a separate access sheath.

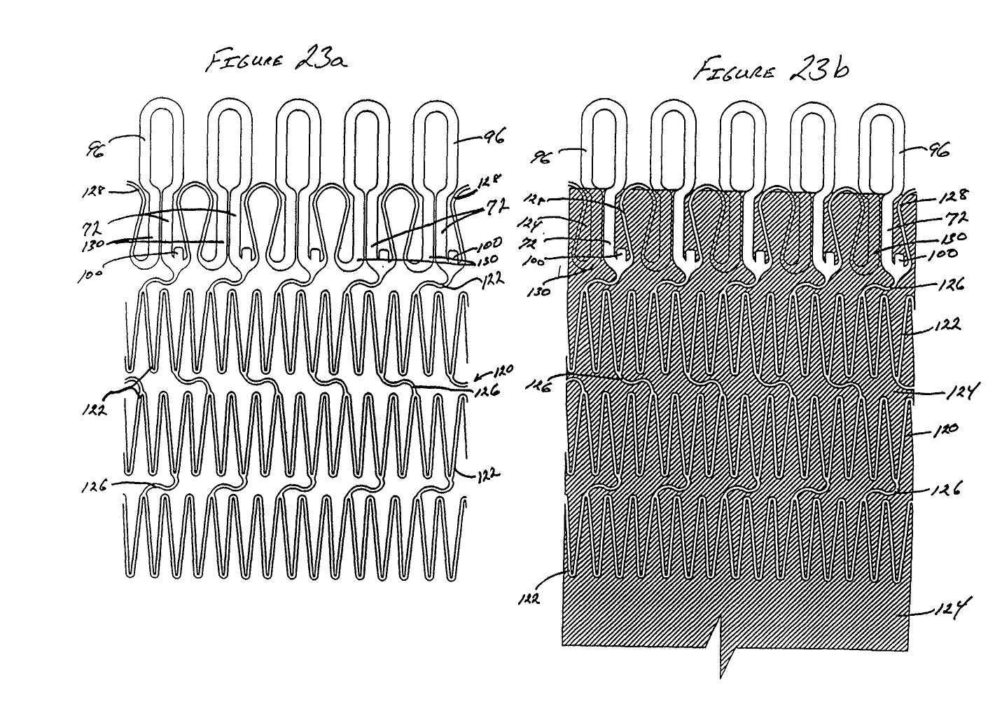

FIG. 21 shows the flattened profile of another sheathless end-side fitting 82. This fitting may be fabricated from a flat sheet, which is rolled and thermally formed into a tubular cross-section, or a tube. The pattern of petals 94, 96, and 98, and tabs 100 are laser cut, chemically etched, or created using another manufacturing process. The petals are then thermally formed into the desired pattern. This fitting incorporates a leading petal 94, a trailing petal 98, and two side petals 96. For small vessels, side petals 96 are curved such that they match the cross-section of the host vessel, leading petal 94 is oriented to extend along the host vessel wall, and trailing petal 98 produces an acute angle relative to the stem of the fitting to ensure leading petal 94 engages the host vessel wall and side petals 96 are appropriately positioned. This sheathless end-side fitting inherently produces a 45 degree angle from the bypass graft to the host vessel and better covers the opening through the host vessel wall when the bypass graft has approximately the same diameter as the host vessel.

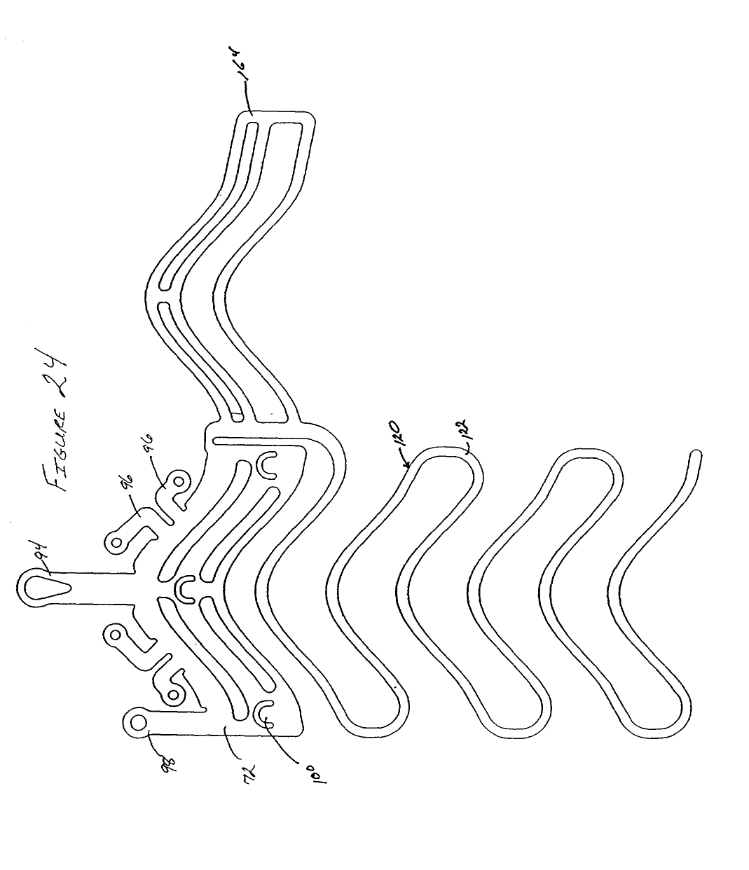

FIG. 22A shows an alternative strain relief 120 embodiment. Instead of coiling wire member 122 of the strain relief into a helix, wire member 122 is shaped into a zig-zag pattern that includes loops 172 connected with straight links 166. This strain relief 120 provides additional column strength to the bypass graft, which may enhance the advancement of the end-side fitting and bypass graft through the loading sheath and into the host vessel. In addition, improving the column strength of the bypass graft provides the operator with enhanced control when manipulating the position of the end-side fitting relative to the host vessel. By separating opposing loops 172, this strain relief may be advanced over the side of the bypass graft when connecting the bypass graft to the end-side fitting. Once positioned, force stressing the loops 172 is removed allowing the strain relief to return towards its preformed configuration around the bypass graft. FIG. 22B shows an alternative strain relief 120 embodiment. This embodiment includes a zig-zap pattern of loops but the links 168 connecting the loops 172 are angled. Angling links 168 provides increased flexibility of the strain relief to enable bending the bypass graft into a tighter radius of curvature without kinking. This strain relief 120 incorporates a coiled retaining member 170 that compresses the bypass graft against the base or stem of the fitting to secure the bypass graft to the end-side fitting.

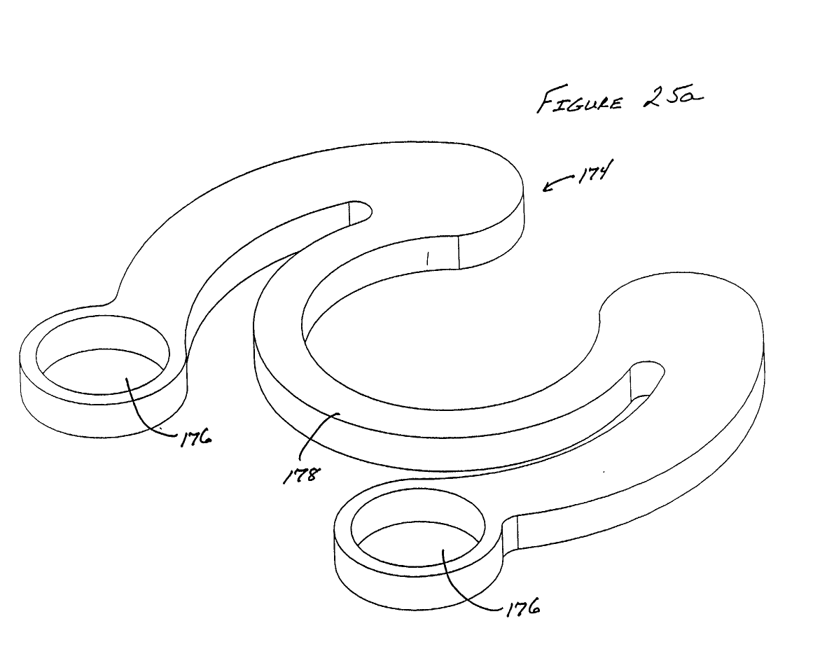

FIGS. 23A and 23B show flattened profiles of an expandable, compressible fitting that incorporates a strain relief 120 integrally to stem 72 of the end-side fitting. The base of the fitting is separated into stem section 72, which is attached to the strain relief, and a radially deflectable section 130. Stem sections 72 and deflectable sections 130 are attached at the side petals 96 and links 128. The radially deflectable section 130 may be compressed and positioned within the interior of the bypass graft; once the bypass graft is positioned over deflectable sections 130 and within stem sections 72, the deflectable section is released thereby compressing the bypass graft against stem sections 72, and securing the bypass graft to the end-side fitting. As such, a separate retaining ring is not required to bond the bypass graft to the base of the fitting. Stem sections 72 incorporate tabs 100 that are preshaped inward to prevent dislodgement of the bypass graft from the base of the fitting.

The base and strain relief in this integrated fitting embodiment can be expanded and compressed either before or after thermally forming into the desired configuration. As such, a single tube stock having the illustrated pattern may be used to address multiple bypass graft diameters by thermally forming the tube stock into the specific diameter. In addition, after thermally forming the end-side fitting, the compressible, expandable nature of the fitting enables addressing a wider range of bypass graft diameters using a single end-side fitting configuration. Finally, compressible, expandable end-side fittings may be compressed into a reduced diameter for insertion through the host vessel wall aperture thereby reducing the required diameters of the loading sheath and access sheath, and the cross-sectional area of the aperture. Reducing the disparity between the outer diameter of the access sheath (or deployment device that determines the maximum expansion of the aperture) and the enlarged outer diameter of the compressible, expandable fitting stem better ensures hemostasis when the end-side fitting is positioned in its enlarged, resting configuration within the host vessel wall aperture.

The compressible, expandable end-side fitting embodiment shown in FIG. 23A may alternatively be used as a composite fitting that includes a strain relief and a collar. The strain relief component of the fitting is positioned on the external surface of the bypass graft as shown in FIG. 23B. Alternatively, the strain relief may be compressed into a reduced diameter and inserted into the bypass graft, at which point it is allowed to expand towards its resting configuration where it contacts the interior surface of the bypass graft (not shown). Either way, the compressible, expandable end-side fitting is secured to the bypass graft and links 128 connecting petals 96 of the fitting are biased outward to function as a collar, as well as the base or stem 72, of the fitting. For the end-side fitting to operate in this manner, the compressible, expandable end-side fitting is preferably positioned around the exterior of the bypass graft and base or stem 72 of the fitting compresses against a separate inner member, over which the bypass graft is placed. Alternatively, strain relief 120 section of the fitting may be modified to incorporate radial extensions that are designed to contact the exterior surface of the host vessel once the petals are positioned against the interior surface of the host vessel.

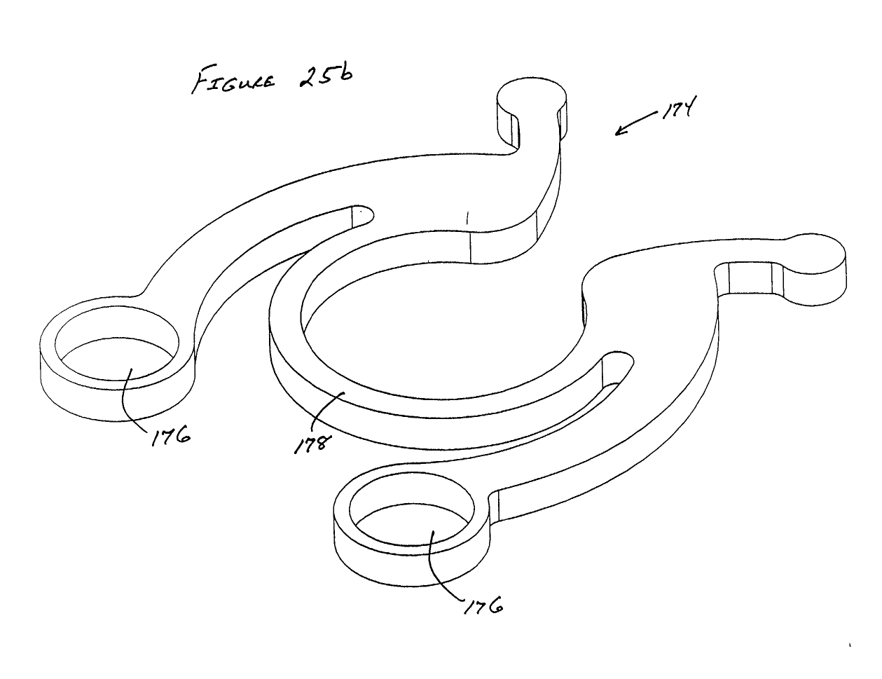

FIG. 24 shows another compressible, expandable end-side fitting that incorporates the strain relief integrally attached to the base of the fitting. This compressible, expandable end-side fitting is a split wall version designed to compress into a reduced diameter as opposite sides of base or stem 72 are coiled. This end-side fitting is fabricated from a flat sheet of superelastic material, is coiled such that base or stem 72 of the fitting, retaining ring member 164, and strain relief 120 have the desired resting diameter and orientation, and is thermally formed into the desired shape. It should be noted that the resting diameter of base or stem 72 of the fitting may be different from the resting diameter of strain relief 120. Strain relief 120 in this embodiment is a zig-zag wire 122 pattern designed to reinforce the bypass graft and enhance column strength as previously described. Incorporated in strain relief 120 is a retaining ring 164 designed to wrap around base or stem 72 and compress the bypass graft against base or stem 72 of the fitting. Tabs 100 are incorporated in base or stem 72 and may be thermally formed in an outward orientation to enhance the attachment of the bypass graft to base or stem 72. The integrated retaining ring 164 is adapted to maintain contact against the bypass graft and continue to compress the bypass graft against base or stem 72 of the fitting as the base or stem is coiled into a reduced diameter. The strain relief 120 in turn is configured to enable coiling into a reduced diameter, if needed.

This compressible, expandable end-side fitting embodiment is adapted to orient the bypass graft at an approximate 45 degree angle relative to the host vessel; other angles may be achieved by changing the flattened profile of the petals. As such, leading petal 94 is designed to be oriented along the host vessel wall; the trailing petal is designed to extend at an acute angle relative to base or stem 72; side petals 96 are configured for small vessels in that they extend a short distance from the base or stem 72 of the fitting and may be thermally formed to match the interior surface of the host vessel.

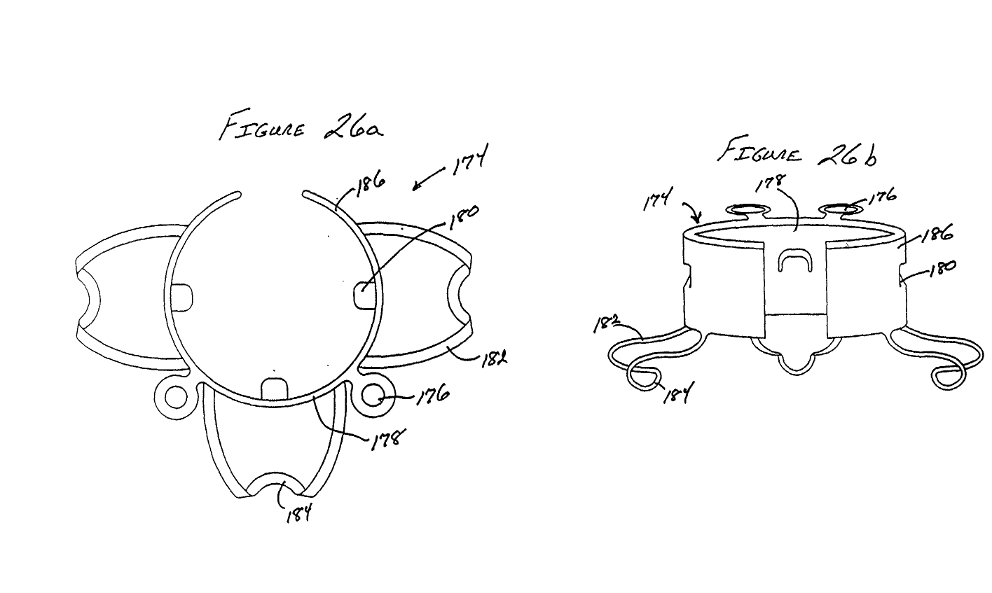

FIGS. 25A and 25B show two collar or compression ring configurations 174 designed secure the end-side fitting to the host vessel wall. Collar 174 includes a gap between opposing sides, a hinge 178 connecting opposing sides, and eyelets 176 incorporated in each opposing side. A clamp may be inserted into eyelets 176 and squeezed together to cause the gap to enlarge. In this configuration, collar 174 may be advanced over the side of the bypass graft and fitting base or stem, and positioned against the exterior surface of the host vessel wall. Once positioned, the clamp is released, allowing the gap to close and cause the collar to compress against the base or stem of the end-side fitting. Once secured to the end-side fitting, the collar maintains the position of the end-side fitting relative to the host vessel wall ensuring the petals of the fitting remain in intimate contact with the interior surface of the host vessel wall.

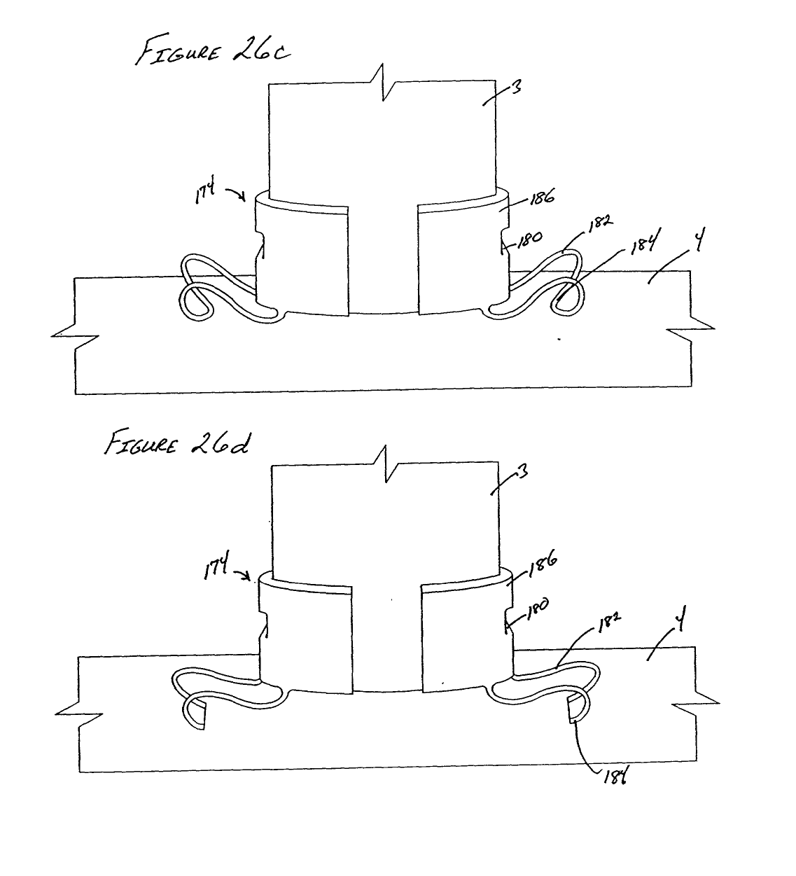

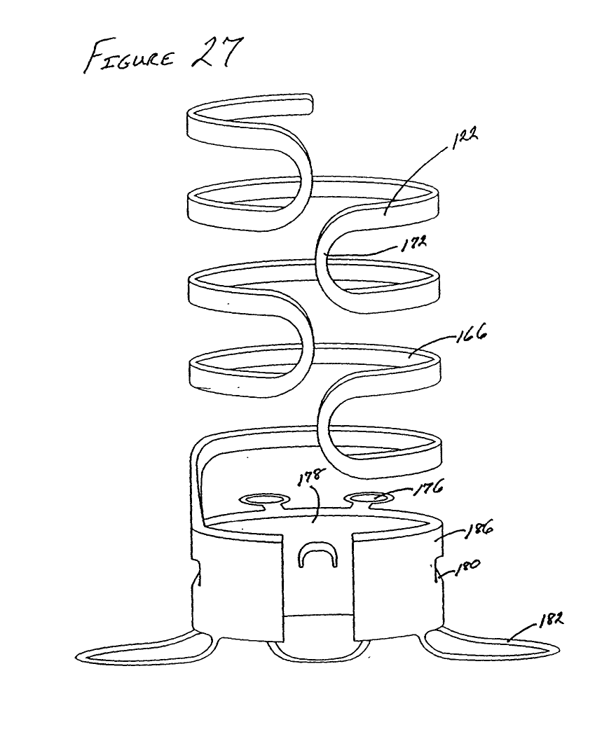

FIGS. 26A and 26B show a top view and a perspective view of an alternative collar embodiment 174 that is designed to compress the vessel wall against the petals of the end-side fitting as well as towards the base or stem of the fitting. Collar 174 incorporates a stem 186 having a hinge 178 around which the gap in the collar may be enlarged. Stem 186 incorporates holders that have eyelets 176 through which a clamp may be temporarily secured and used to enlarge the gap. Stem 186 incorporates tabs 180 that are biased inward to prevent axial dislodgement of the collar 174 once positioned over the base or stem of the end-side fitting and bypass graft. Extensions 182 emanate away from stem 186 of collar 174 and incorporate distal protrusions 184 designed to gather tissue once positioned. Extensions 182 act similar to the petals of the end-side fittings previously described in that they may be deflected during positioning and return towards their preformed configuration once positioned against the exterior surface of the host vessel wall.

FIGS. 26C and 26D show representative steps of positioning the collar. Extensions 182 are deflected away from the distal end of collar stem 186 back towards the top, or proximal end, of collar 174. This may be performed with a secondary operation than enlarging the gap in the collar or may occur as a result of enlarging the gap. By squeezing eyelets 176 together, the gap is enlarged and the inherent curve extensions 182 emanate from stem 186 causes extensions 182 to deflect upwards toward the proximal end of the collar, as shown in FIG. 26C. Once the collar is positioned around the bypass graft and base or stem of the end-side fitting, and engages the exterior surface of the host vessel wall, the external force(s) causing the gap to open and the extensions to deflect is removed. This causes the extensions to return towards their preformed configuration and urge extension tabs 184 into contact with the host vessel wall. Extension tabs 184 gather the host vessel wall toward the base or stem of the fitting providing improved hemostasis at the opening through the host vessel wall where the end-side fitting and bypass graft exit the host vessel.

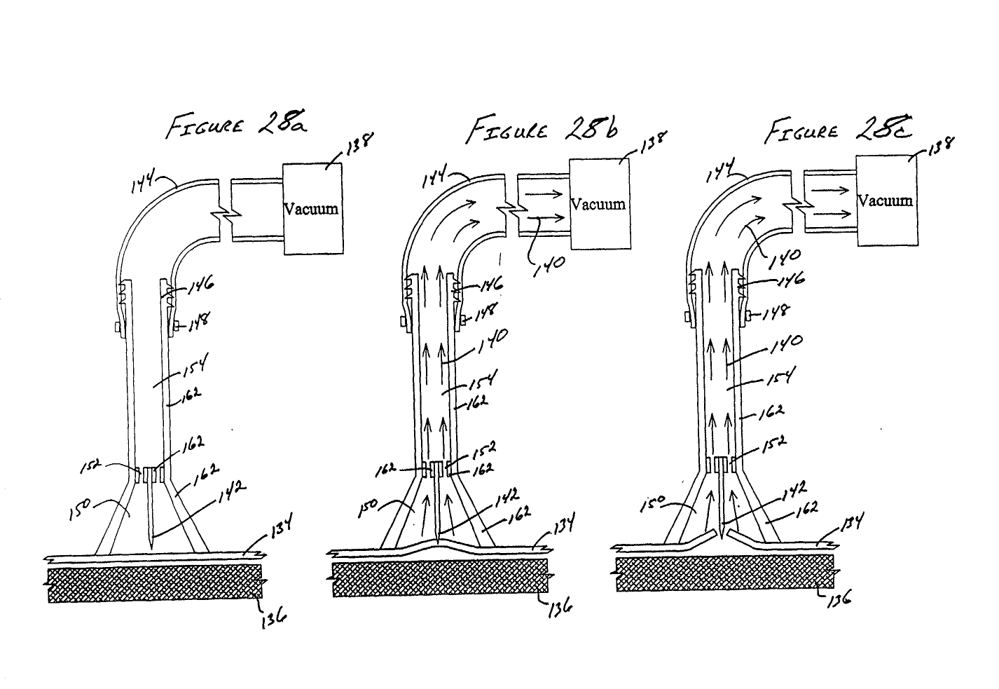

FIG. 27 shows a perspective view of a strain relief that incorporates a collar. The strain relief/collar has a stem designed to compress the bypass graft against the base or stem of the end-side fitting. Tabs 180 prevent axial dislodgement of the strain relief/collar from the end-side fitting. Eyelets 176 provide a structure that a clamp or other surgical instrument may be used to open the gap in stem 186 to position the stem around the bypass graft and end-side fitting. The strain relief/collar incorporates a wire member looped in a zig-zag pattern to provide column strength and axial flexibility to prevent kinking or over-expansion of the bypass graft away from the anastomosis site. The strain relief/collar incorporates extensions 182 that may be deflected back towards stem 186 for advancing through the deployment system and return towards their preformed configuration once the end-side fitting is positioned. Extensions 182 engage the exterior surface of the host vessel wall and ensure the petals of the end-side fitting maintain intimate contact with the interior surface of the host vessel wall.

Stem 186 of the strain relief/collar embodiment described above and shown in FIG. 27 is a split wall design incorporating tabs 180 and a gap between opposing sides. The stem may alternatively consist of a wire wound into a helix, looped similar to the strain relief section of the device, or formed into a double helix, mesh, or other configuration. These stem configurations extend from strain relief wire 122 and are biased to form extensions 182 that convert the strain relief into a combination strain relief and collar.

Additional Less Invasive Tools

The deployment and fitting embodiments described above illustrate devices that create anastomoses between bypass grafts and host vessels in a less invasive manner than conventional techniques. Those devices primarily focus on attaching the bypass graft and do not address identifying and adequately exposing the host vessel, which may itself be time consuming.

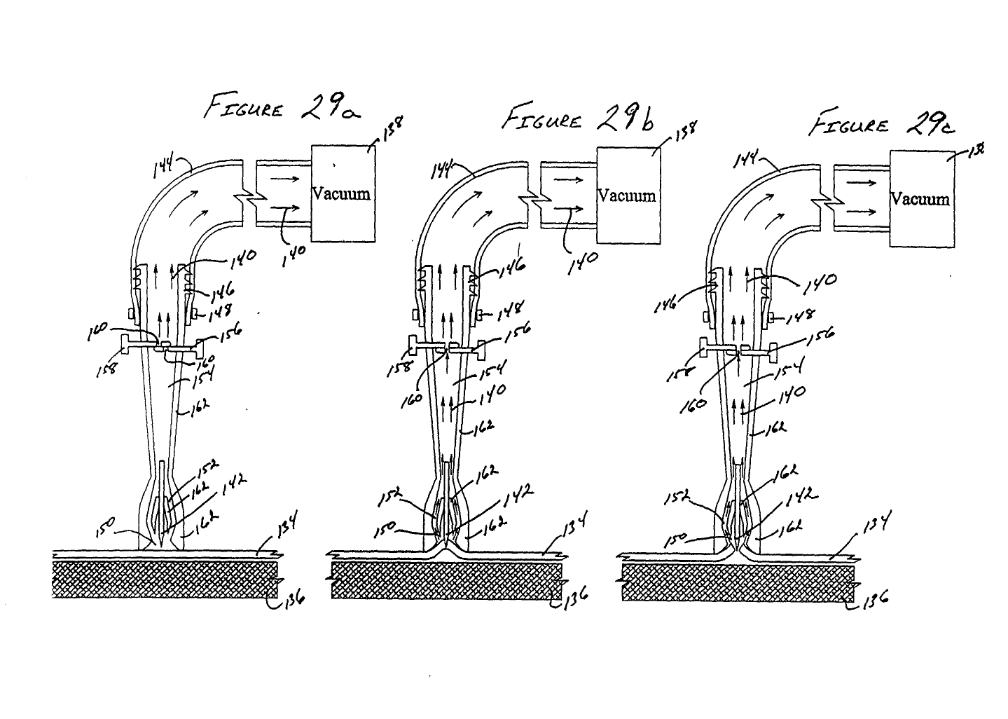

FIGS. 28A to 28C illustrate a vacuum incising device designed to accurately create an incision in a first layer of tissue 134, which directly contacts a second tissue layer 136 that the operator must not damage. For example, first layer of tissue 134 may be the parietal pericardium and the second layer of tissue 136 may be the anatomy of the heart including the coronary vessels. To access the pericardial space, the parietal pericardium must be cut and the incision opened. During open heart procedures, the pericardium is subsequently cradled to support the heart. This process of cutting the parietal pericardium without damaging the underlying structures becomes more difficult as the procedures become less invasive. For example, endoscopic procedures are associated with limited motion through the access ports. As such, grasping and cutting the parietal pericardium using separate instruments is delicate and is associated with a relatively high morbidity of cutting unwanted anatomy.

The vacuum incising device in FIGS. 28A to 28C is designed to controllable isolate a layer of tissue and cut that layer using a single instrument, making this step amenable to less invasive approaches. The vacuum incising device incorporates a funnel-shaped distal structure to isolate the region of tissue the operator intends to cut. The funnel-shaped distal structure is integral with main body 162 of the vacuum incising device. As such this device may be injection molded as a single unit. A scalpel blade 142 (or other incising mechanism) is attached to the device such that the distal tip of the blade is offset from the end of the funnel-shaped distal structure. As such the distal tip of the blade does not contact tissue when the funnel-shaped distal structure abuts the layer of tissue, and no vacuum is applied, as shown in FIG. 28A. Lumens 152 are dispersed between attached blade 142 and the inner diameter of device main body 162. Lumens 152 provide a conduit from the interior of main body 154 and into cavity 150 of the distal structure to produce suction within cavity 150 of the funnel-shaped distal structure. Tubing 144 is routed between proximal locking connector 146 of the vacuum incising device and vacuum source 138. As FIG. 28B shows, applying a vacuum causes the first layer of tissue 134 to pull away from the second layer of tissue 136 and within the funnel-shaped distal structure, and engage the tip of blade 142. As FIG. 28C shows, the layer of tissue is cut as the vacuum pulls the tissue past the tip of the blade. This vacuum incising device is particularly suitable for cutting a discrete length of tissue.

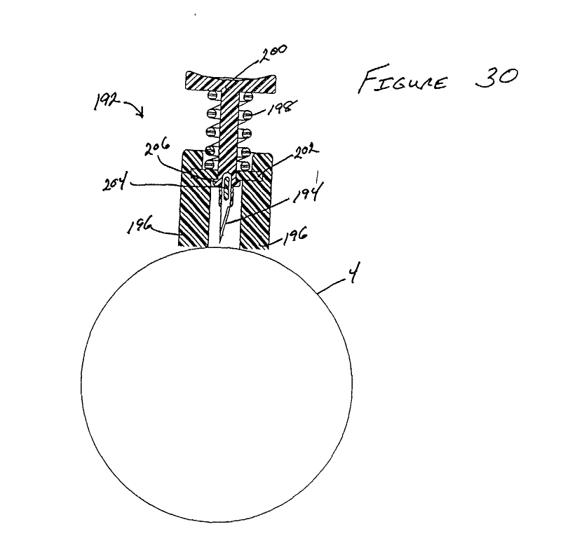

FIGS. 29A to 29C show an alternative vacuum incising device capable of being dragged along a tissue layer to produce a long incision. Opposing halves of a mechanically actuated valve mechanism 156 and 158 determine whether suction is applied at the distal structure. In the separated orientation, lumens 160 of the opposing valve halves 156 and 158 do not overlap; therefore, no suction is applied (as shown in FIG. 29A). When opposing valve halves 156 and 158 are squeezed together, however, lumens 160 overlap and suction is applied (as shown in FIGS. 29B and 29C). A spring mechanism may be incorporated in valve mechanisms 156 and 158 to ensure lumens 160 do not overlap in the resting configuration. This prevents unwanted cutting of the tissue layer.

As FIGS. 29B and 29C show, the applied suction causes the contacted layer of tissue 134 to engage against scalpel blade 142 thereby cutting the layer of tissue 134. This vacuum incising device may be dragged along first layer of tissue 134 in order to cut a length thereof.

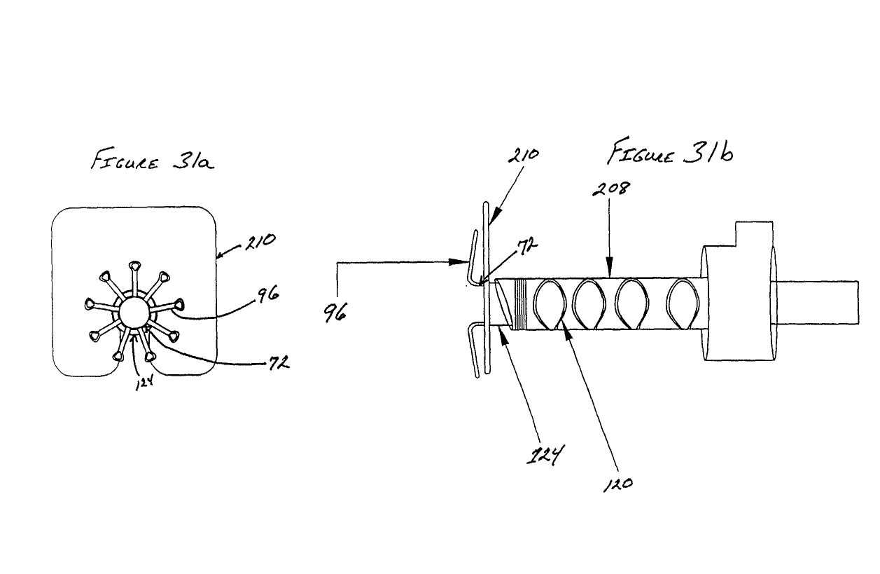

FIG. 30 shows a spring loaded incising device capable of creating a cut through host vessel wall 4. A standard scalpel blade 194 may be attached to incising device 192 by placing over blade holder 204, characteristic of conventional scalpels. Stabilizing legs 196 maintain the position of incising device 192 relative to the host vessel wall 4 during the cutting process. A handle knob 200 is attached to blade holder 204, thus scalpel blade 194, and is used to advance the blade 194 through the host vessel wall 4 at a known distance. A spring 198 is attached to handle knob 200 and a spring holder 202, which is attached to stabilizing legs 196. Spring 198 urges handle knob 200 away from the spring holder 202 so the resting position of incising device 192 is configured such that blade 194 is retracted within the distal end of stabilizing legs 196. The blade holder 204 incorporates a stop 206 that limits the proximal motion of blade holder 204, thus blade 194.

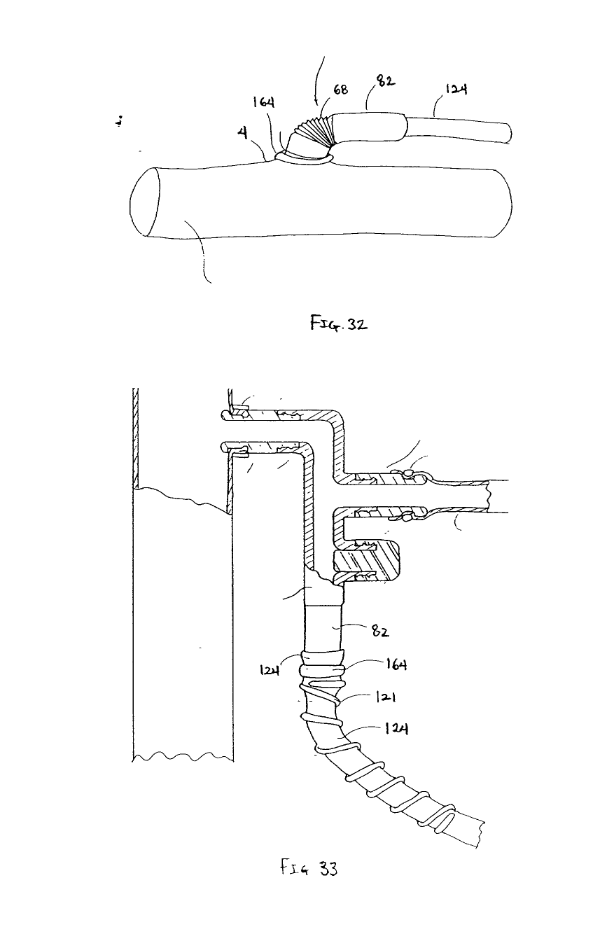

FIGS. 31A and 31B show a loading aid designed to facilitate positioning strain relief 120 over bypass graft 124, and base or stem 72 of the end-side fitting. Once positioned, strain relief 120 returns towards its preformed configuration providing a compression force against the bypass graft to secure the bypass graft to the base or stem of the fitting. Strain relief 120 is expanded into an enlarged diameter and housed around a loading sheath 208. Once the bypass graft 124 is advanced over base or stem 72 of the end-side fitting, loading sheath 208 is positioned over the bypass graft and is located along the stem such that the distal end of loading sheath 208 abuts the position along base or stem 72 of the fitting where the distal end of strain relief 120 is designed to reside.

Once loading sheath 208 is positioned, strain relief 120 is advanced; once strain relief 120 extends beyond the distal end of loading sheath 208 the strain relief returns towards its preformed, reduced diameter configuration. If the distal end of the strain relief is advanced too far towards petals 96 of the fitting, the strain relief does not seat on the base or stem of the fitting and interferes with compressing petals 96 into a reduced diameter for deployment. Therefore, a loading aid 210 may be used.

Loading aid 210 consists of a sheet of polymer, metal, or metal alloy material having a hole to match the base or stem of the fitting and a side notch along which base or stem 72 of the fitting may be inserted or removed. Loading aid 210 is positioned against petals 96 and has a large enough wall thickness to locate the distal end of strain relief 120 at desired location along base or stem 72 of the end-side fitting.

This invention has been described and specific examples of the invention have been portrayed. The use of those specific examples is not intended to limit the invention in any way. Additionally, to the extent that there are variations of the invention which are within the spirit of the disclosure and yet are equivalent to the inventions found in the claims, it is our intent that those claims cover those variations as well.

We claim:

1. An anastomosis connector system for providing support to a bypass graft having a wall comprising:
a fitting adapted for insertion at least partially through a vessel wall at an attachment site, the fitting being attachable to a distal end of the bypass graft;
an elongate member having a proximal end and a distal end with a length therebetween, wherein the member extends at least partially about the bypass graft at a location along the graft proximal to the attachment site between the distal end of the graft and the vessel wall such that kinking is inhibited within the graft; and
a collar for compressing the distal end of the graft against the fitting.

2. The system of claim 1 wherein the elongate member comprises a wire.

3. The system of claim 1 wherein the elongate member is wound into a helical pattern about the bypass graft.

4. The system of claim 1 wherein the elongate member is further adapted to increase a burst strength of the bypass graft.

5. The system of claim 1 wherein the elongate member is incorporated into the wall of the bypass graft.

6. The system of claim 5 wherein the bypass graft comprises a synthetic material.

7. The system of claim 1 wherein the elongate member is adapted to be disposed exteriorly to the bypass graft.

8. The system of claim 1 wherein each of the proximal and distal ends of the elongate member are bonded to the fitting.

9. The system of claim 1 wherein the distal end of the elongate member is stiff relative to the proximal end of the elongate member such that the length is tapered in stiffness from the distal to the proximal end.

10. The system of claim 1 wherein the elongate member extends 15 turns about the bypass graft.

11. The system of claim 1 wherein the elongate member is wound in a zig-zag pattern about the bypass graft.

12. The system of claim 11 wherein the zig-zag pattern comprises a plurality of loops connected by a plurality of corresponding links.

13. The system of claim 12 wherein the links connecting the loops are straight relative to each other.

14. The system of claim 12 wherein the links connecting the loops are angled relative to each other.

15. The system of claim 1 wherein the elongate member further comprises a coiled retaining member.

16. The system of claim 1 wherein the elongate member is integrally attached to the fitting.

17. The system of claim 16 wherein the fitting comprises a base having a plurality of radially extendable petals, each of the petals being attached to the base by a stem section and a radially deflectable section adapted to be positioned within an interior of the graft while compressing a portion of the graft between the stem section and the deflectable section.

18. The system of claim 17 wherein the stem section further comprises a tab adapted to compress against the graft.

19. The system of claim 1 wherein the fitting further comprises an integral retaining ring adapted to wrap around the fitting and compress the distal end of the graft against the fitting.

20. The system of claim 1 wherein the elongate member extending about the graft is adapted to orient the graft at an angle relative to a longitudinal axis defined along the vessel wall.

21. The system of claim 20 wherein the angle is about 45°.

22. The system of claim 1 wherein the collar comprises a pair of opposing members movable about a hinge, wherein the collar further defines a gap which is variably enlargable corresponding to movement of the members.

23. The system of claim 22 wherein each of the opposing members define an eyelet.

24. The system of claim 22 wherein the collar further comprises a plurality of extensions extending away from the collar.

25. The system of claim 24 wherein each of the extensions further comprise a protrusion adapted to engage an exterior surface of the vessel wall.

26. The system of claim 1 wherein the elongate member further comprises a collar integrally connected at the distal end of the elongate member.

27. The system of claim 26 wherein the collar is adapted to compress the distal end of the graft against the fitting.

28. The system of claim 26 wherein the collar comprises a pair of opposing members movable about a hinge, wherein the collar further defines a gap which is variably enlargable corresponding to movement of the opposing members.

29. The system of claim 1 wherein the fitting comprises a material selected from the group consisting of stainless steel, titanium, nickel-titanium alloy, thermoplastic, thermoset plastic, silicone, and combinations thereof.

30. The system of claim 1 wherein the elongate member comprises a material selected from the group consisting of stainless steel, shape memory alloy, and polymer.

31. The system of claim 30 wherein the shape memory alloy comprises nickel titanium.

32. The system of claim 30 wherein the polymer comprises nylon or polyester.

33. The system of claim 1 wherein the collar comprises a material selected from the group consisting of polyethylene, polyurethane, polycarbonate, thermoplastic, silicone, nickel titanium, spring stainless steel, and combinations thereof.
